(19) United States
(12) Patent Application Publication
(10) Pub. No.: US 2025/0372251 A1
(43) Pub. Date: Dec. 4, 2025

(54) ARTICLES AND METHODS FOR FORMAT INDEPENDENT DETECTION OF HIDDEN CARDIOVASCULAR DISEASE FROM PRINTED ELECTROCARDIOGRAPHIC IMAGES USING DEEP LEARNING

(71) Applicant: Yale University, New Haven, CT (US)

(72) Inventors: Rohan Khera, New Haven, CT (US); Veer Sangha, New Haven, CT (US)

(21) Appl. No.: 18/870,024

(22) PCT Filed: May 26, 2023
(86) PCT No.: PCT/US2023/023729

§ 371 Date: Nov. 27, 2024

Related U.S. Application Data
(60) Provisional application No. 63/346,610, filed on May 27, 2022.

(51) Int. Cl. G16H 50/20; A61B 5/00; A61B 5/02; A61B 5/338; A61B 5/346; G06V 10/82; G16H 30/40

(52) U.S. Cl. CPC G16H 50/20; A61B 5/02028; A61B 5/338; A61B 5/346; A61B 5/7267; G06V 10/82; G16H 30/40

(57) ABSTRACT

Provided herein are computer-implemented methods of detecting cardiovascular disease in a subject. The methods include receiving an electrocardiogram (ECG) image for the subject; applying a machine-learning based algorithm to the ECG image for the subject, the algorithm being trained to distinguish a printed ECG reading of a heart with cardiovascular disease from a printed ECG reading of a healthy heart; comparing outputs of the algorithm to patterns of algorithm outputs for ECG images from healthy subjects and subjects with one or more cardiovascular diseases; and determining if the subject has cardiovascular disease based upon the outputs of the algorithm.

### CROSS-REFERENCE TO RELATED APPLICATIONS

The present application claims priority under 35 U.S.C. § 119(e) to U.S. Provisional Patent Application No. 63/346,610, filed May 27, 2022, which application is incorporated herein by reference in its entirety.

### BACKGROUND OF THE INVENTION

Cost effective drug and device therapy can improve prognosis for patients with cardiovascular disorders, but early detection and initiation of therapy is key. Many cardiovascular disorders remain undiagnosed or hidden until they manifest as clinical disease. Screening for these cardiovascular diseases can detect them early, but primarily occurs in using advanced diagnostic tools, such as echocardiography, CT, or MRI, all of which are costly and have high barriers to use for screening in the general population.

Electrocardiography is a relatively low cost and easy to obtain tool in the diagnosis and management of cardiovascular disease. Screening algorithms for hidden disorders including Left Ventricular Systolic Dysfunction, Cardiomyopathies, Aortic Stenosis, and Pulmonary Hypertension based on electrocardiograms have been proposed by several groups, with recently published Artificial Intelligence (AI) based algorithms developed for 12-lead and single lead ECG voltage signal data displaying performance that might qualify them for use as a screening tool for high risk patients. These algorithms are trained and make predictions on raw electrocardiographic signals collected from machines, not from the printed waveforms that are commonly interpreted and used by trained clinicians. This reliance on signal-based models reduces the accessibility of these models as a method of screening to be conducted in family practice clinics, emergency rooms, and remote settings. Additionally, existing models for hidden cardiovascular label detection are trained and tested on data from a single source, with an inability to infer broad generalizability to different institutions and health settings, and also lack interpretability measures that provide information that can be interpreted by humans on features in the electrocardiogram relevant to the prediction derived from the model.

Accordingly, there is a need in the art for articles and methods that improve on existing detection methods by providing early screening utilizing printed waveforms, independent of their format. The present invention addresses this need.

### SUMMARY OF THE INVENTION

In one aspect, a computer-implemented method of detecting cardiovascular disease in a subject includes receiving an electrocardiogram (ECG) image for the subject; applying a machine-learning based algorithm to the ECG image for the subject, the algorithm being trained to distinguish a printed ECG reading of a heart with cardiovascular disease from a printed ECG reading of a healthy heart; comparing outputs of the algorithm to patterns of algorithm outputs for ECG images from healthy subjects and subjects with one or more cardiovascular diseases; and determining if the subject has cardiovascular disease based upon the outputs of the algorithm.

In some embodiments, the machine-learning based algorithm is a deep neural network, the deep neural network comprising a plurality of nodes trained to distinguish a printed ECG reading of a heart with cardiovascular disease from a printed ECG reading of a healthy heart. In some embodiments, the machine-learning based algorithm is another machine learning-based algorithm or a statistical algorithm.

In some embodiments, the ECG image comprises a printed ECG image of an ECG dataset formed by conversion of ECG waveform data. In some embodiments, the method is generalizable to multiple ECG image formats. In some embodiments, the algorithm is trained on ECG images having incorrectly placed leads. In some embodiments, the algorithm is trained on images of ECGs with different signal, background, and noise characteristics. In some embodiments, the method further includes identifying hidden clinical labels. In some embodiments, the method further includes identifying characteristics of the ECG image that the determination is based on. In some embodiments, the method is automated.

In some embodiments, the cardiovascular disease comprises a disorder selected from the group consisting of structural disorders of the heart, functional disorders of the heart, structural disorders of the structures supporting the heart, functional disorders of the structures supporting the heart, and combinations thereof. In some embodiments, the disorder comprises abnormalities of the muscle, valves, blood vessels, or lining of the heart. In some embodiments, the disorder is a genetic disorder. In some embodiments, the disorder is an acquired disorder. In some embodiments, the cardiovascular disease comprises a disease that is not normally discernable by physicians from ECG data.

In some embodiments, prior to the step of applying the algorithm to the ECG image for the subject, the method further includes training the algorithm, the training of the algorithm including creating an image-based dataset including a normal subset and a cardiovascular disease subset; optionally pre-training the algorithm on an unrelated clinical or hidden label; and training the algorithm on the image-based dataset. In some embodiments, the cardiovascular disease subset includes a low ejection fraction (EF) subset. In some embodiments, the low EF subset includes ECG images for individuals with EF of less than 40%. In some embodiments, the clinical label includes six physician-defined labels and the hidden label includes gender. In some embodiments, the normal subset includes ECG images for individuals having hypertrophic cardiomyopathy (HCM). In some embodiments, the cardiovascular disease subset includes ECG images for individuals having HCM and left ventricular (LV) systolic dysfunction. In some embodiments, the image-based dataset includes at least two different plotting schemes for each ECG waveform. In some embodiments, the image-based dataset includes at least two different ECG image formats.

### BRIEF DESCRIPTION OF THE DRAWINGS

FIG. 1 shows a schematic illustrating a study outline. Panel A shows data processing, panel B shows model training, and panel C shows model validation. Abbreviations: ECG, electrocardiogram; EF, ejection fraction; FC, fully connected layers; Grad-CAM, gradient-weighted class activation mapping; CT, Connecticut; ELSA-Brasil, Estudo Longitudinal de Sande do Adulto (The Brazilian Longitudinal Study of Adult Health); MO, Missouri; TX, Texas.

FIG. 2 shows a schematic illustrating a flow chart of study cohort and analysis.

FIG. 3 shows schematics illustrating EfficientNet-B3 architecture used for model development. Our image-based convolutional neural network (CNN) is designed Efficientnet-B3 architecture to recognize visual patterns of LV systolic dysfunction from ECG-images. The input layer receives the ECG image as a matrix of pixel values. The convolutional layer applies a set of learnable filters to the input image that slide across the image, convolving it and producing an output feature map. These filters detect different features, such as edges, shapes, or textures, that are important for identifying patterns in the ECG image. The pooling layer reduces the spatial size of the output feature maps by performing a down-sampling operation, which helps to make the model more robust to variations in the image. Finally, the output layer produces the prediction of LV systolic dysfunction from the image.

FIGS. 4A-D show graphs illustrating Novel ECG image formats. (A) standard format with lead I as rhythm strip, (B) three-rhythm format with leads I, II, and V1 as rhythm strips, (C) no-rhythm format with no rhythm strip, and (D) rhythm on top with lead I as rhythm strip located on top. Standard format was used both in model training and validation and is presented for comparison. The three other layouts were only used for validation to assess model performance on image formats not encountered before.

FIGS. 5A-C show graphs illustrating ECG calibrations in validation studies. Model performance was assessed across various ECG calibrations at (A) 10 mm/mV, (B) 5 mm/mV, and (C) 20 mm/mV.

FIG. 6 shows a graph illustrating representative image of a real-world electrocardiogram from Cedars Sinai Medical Center, Los Angeles, CA used for validation.

FIGS. 7A-B show graphs illustrating representative examples of real-world electrocardiograms from (A) outpatient clinics of Yale New Haven Hospital (YNHH), and (B) Lake Regional Hospital (LRH) used for validation.

FIGS. 8A-C shows graphs illustrating representative examples of real-world electrocardiograms from Memorial Hermann Health System in Houston, TX used for external validation.

FIGS. 9A-E show graphs illustrating representative examples of real-world electrocardiograms from Methodist Cardiology Clinic in San Antonio, TX for external validation.

FIGS. 10A-C show graphs illustrating model performance measures. (A) Receiver-operating curve. (B) Precision-recall curve. (C) Diagnostic odds ratios.

FIGS. 11A-B show graphs illustrating proportion of individuals with LV systolic dysfunction and mean LV ejection fraction across deciles of predicted probability of LV systolic dysfunction. (A) Proportion of individuals with LV Systolic Dysfunction across deciles of model-predicted probabilities of LV Systolic Dysfunction. (B) Mean LV ejection fraction across deciles of predicted probability of LV Systolic Dysfunction.

FIG. 12 shows a graph illustrating cumulative hazard curves for incident LV systolic dysfunction in model-predicted positive and negative screens amongst the members of the held-out test set with LVEF≥40% and at least one follow-up measurement.

FIGS. 13A-D show graphs illustrating gradient-weighted class activation mapping (Grad-CAMs) across ECG formats. (A) Standard format. (B) Two rhythm leads. (C) Standard shuffled format. (D) Alternate format. The heatmaps represent averages of the 100 positive cases with the most confident model predictions for LVEF<40%.

FIG. 14 shows a graph illustrating distribution of mean Grad-CAM signal intensities in the V2-V3 region (blue) and the other regions (orange) of the ECGs in the top 100 most confident predictions of LV systolic dysfunction. To define the V2-V3 region of interest, the portion of the Grad-CAM output corresponding to elements 6-8 on the horizontal axis and 3-8 on the vertical axis on standard format were selected. Abbreviation: Grad-CAM, Gradient-weighted class activation mapping.

FIGS. 15A-D show graphs illustrating examples of Gradient-weighted Class Activation Mapping (Grad-CAM) analysis of electrocardiograms from four individuals with positive (A and B) and negative (C and D) model predictions for left ventricular systolic dysfunction. Class-discriminating signals localize to anterior leads in positive cases (A and B).

FIGS. 16A-D show graphs illustrating representative examples of Gradient-weighted Class Activation Mapping (Grad-CAM) analysis of electrocardiograms from each validation center (A) outpatient clinics of Yale New Haven Hospital (YNHH), (B) Lake Regional Hospital (LRH), (C) Memorial Hermann Health, and (D) Methodist Cardiology Clinic.

FIG. 17 shows a graph illustrating receiver-operating curves for external validation sites. Abbreviations: AUROC, area under receiver-operating characteristic curve; EF, Ejection fraction; LRH, Lake Regional Hospital; YNHH, Yale New Haven Hospital.

FIGS. 18A-B show graphs illustrating preprocessing of ECG images in electronic PDF format. Representative images for segmentation and quality standardization of electrocardiograms from two patients with (A) LVEF 19%, and (B) LVEF 59%. This preprocessing step removes the peripheral elements of ECG tracing, including annotation and patient identifiers. The corresponding predicted probabilities of LV systolic dysfunction after preprocessing were 0.927 (for the patient with low LVEF), and 0.005 (for the individual with normal LVEF).

FIG. 19 shows images illustrating preprocessing of ECG photographs obtained by a smartphone. Images represent segmentation and quality standardization of ECG photographs with extreme variations of photo rotations, shadows, and skew angles. Photos were obtained by iPhone 12 from electrocardiograms of a patient with LVEF of 19% before and after segmentation and quality standardization with corresponding model predictions for LV systolic dysfunction.

FIG. 20 shows images illustrating preprocessing of ECG photographs obtained by a smartphone. Images represent segmentation and quality standardization of ECG photographs with extreme variations of photo rotations, shadows, and skew angles. Photos were obtained by iPhone 12 from electrocardiograms of a patient with LVEF of 59% before and after segmentation and quality standardization with corresponding model predictions for LV systolic dysfunction.

FIGS. 21A-B show graphs illustrating changes in Model Predictions with proportional variations in (A) brightness, and (B) contrast. The model-predicted probability from the unaltered image with original brightness and contrast was considered as the reference.

FIGS. 22A-B show graphs illustrating ECG images from an individual with LVEF of 31.0%. (A) original image with variations in contrast and brightness, (B) changes in model predictions with variation in contrast, and (C) changes in model predictions with variation in brightness.

FIGS. 23A-B show graphs illustrating ECG images from an individual with LVEF of 57.4%. (A) original image with variations in contrast and brightness, (B) changes in model predictions with variation in contrast, and (C) changes in model predictions with variation in brightness.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

Provided herein are articles and methods for detecting cardiovascular diseases and/or predicting their future risk from printed electrocardiograms. In some embodiments, the method is a computer-implemented method of detecting cardiovascular disease in a subject, the method including receiving a printed electrocardiographic (ECG) reading for a subject, applying a machine-learning based algorithm, such as a deep neural network, to the ECG image for the subject, and determining if the subject has cardiovascular disease based upon the outputs of the machine-learning based algorithm. In some embodiments, the algorithm is trained to distinguish a printed ECG reading of a heart with one or more cardiovascular diseases from a printed ECG reading of a healthy heart. For example, the deep neural network may include a plurality of nodes trained to distinguish a printed ECG reading of a heart with one or more cardiovascular diseases from a printed ECG reading of a healthy heart. In such embodiments, the method also includes comparing outputs of the nodes from the ECG image for the subject to patterns of node outputs for ECG images of healthy subjects and subjects with one or more cardiovascular diseases. The determining step is then based upon the comparison of the outputs of the nodes.

Although described herein primarily with respect to a deep neural network including a plurality of nodes, as will be appreciated by those skilled in the art, the disclosure is not so limited and may include any other suitable machine learning-based or other statistical method. Such embodiments are expressly considered herein, and include training an algorithm (or providing a trained algorithm) according to any of the embodiments for training the deep neural network, and applying the algorithm to the ECG image for the subject.

In some embodiments, the nodes of the deep neural network are trained prior to the step of applying the deep neural network to the ECG image for the subject. The training of the nodes includes creating a series of image-based datasets with varying ECG lead layouts, optionally pre-training the nodes on a pre-defined set of labels, and then training the nodes on the image-based dataset. The pre-defined set of labels includes any suitable set of labels involved in distinguishing diseased hearts from healthy hearts. The image-based dataset includes a normal subset and a diseased subset, with the diseased subset including ECG images from subjects with any suitable cardiovascular disease for detection with the presently disclosed methods.

The ECG images for the subject and/or training of the nodes includes any suitable ECG image format. In some embodiments, for example, the ECG images include digital images, screenshots, smartphone photos, scans, and/or printed images of partial and/or whole ECGs. In some embodiments, the partial and/or whole ECGs are ECG datasets developed by conversion of the ECG waveform data. The ECG waveform data may include signal data from any suitable number of leads (e.g., 12-lead ECG signal data), stored in any suitable format, and/or from any suitable institution or source. In some embodiments, the image-based dataset includes multiple different plotting schemes for each signal waveform recording. For example, in some embodiments, the image-based dataset includes at least two, at least three, at least four, at least five different plotting schemes for each signal waveform recording, or any suitable combination, sub-combination, range, or sub-range thereof. By utilizing different plotting schemes for each signal waveform recording in the image-based dataset, the deep neural network is able to detect cardiovascular disease in multiple ECG formats. The image-based dataset may also include data collected and stored from different machines and/or at different frequencies and evaluate cardiac disease across a health system.

Additionally, or alternatively, in some embodiments, the image-based dataset includes ECG images having incorrectly placed leads, which enables the deep neural network to detect cardiovascular disease in a manner that is independent of the format of the ECG image presented to the network. The multiple formats and/or incorrectly placed leads teach the deep neural network to identify individual leads on varying ECG formats, such that the deep neural network is able to rely upon lead-specific cues in the ECG images. Accordingly, in some embodiments, the method is generalizable to multiple ECG image formats (i.e., can detect diseases independent of the ECG printed format and in image formats that are not explicitly included in the image-based dataset) and/or able to detect cardiovascular disease in subjects with ECG images produced from incorrectly placed leads.

Additionally, in some embodiments, image-based datasets include ECG images having differences in characteristics. These include but are not limited to differences in cropping, brightness, contrast, color, background color, background line width and characteristics, ECG signal line width and characteristics, and lead label placement, font, and size. These differences teach the deep neural network to identify features in ECGs irrespective of characteristics and qualities of the uploaded image. Accordingly, in some embodiments, the method is generalizable to ECGs that are acquired via smartphone or other device cameras, or via scans.

Suitable cardiovascular diseases for detection with the presently disclosed methods include, but are not limited to, structural disorders of the heart and/or structures supporting the heart, functional disorders of the heart and/or structures supporting the heart, or a combination thereof. Such disorders may arise from abnormalities of the muscle, valves, blood vessels, and/or the lining of the heart, and may be due to genetic causes, environmental causes, lifestyle causes, unknown precipitants of the disease, or combinations thereof. For example, in some embodiments, the disease includes low ejection fraction (EF) of the left ventricle (LVEF), where low EF includes any EF of less than 40%. In such embodiments, the image-based dataset includes a subset with normal EF (i.e., normal subset) and a subset with low EF (i.e., diseased subset). Other suitable diseases include, but are not limited to, left or right ventricular systolic dysfunction, left ventricular diastolic dysfunction, right-sided heart failure, aortic and mitral valve disease, including their stenosis or regurgitation, cardiomyopathy and its various subtypes, pulmonary hypertension, as well as other rare genetic cardiac disorders.

In some embodiments, the cardiovascular disease includes a disease that is not normally discernable by physicians from ECG data. For example, in some embodiments, the deep neural network detects a cardiovascular disease present in a patient at the time of the ECG reading. Additionally, or alternatively, in some embodiments, the deep neural network identifies characteristics of the ECG image that the determination (e.g., disease or no disease) is based on using interpretability tools, including, but not limited to gradient class activation maps that identify regions of the image weighed heavily in the prediction. Accordingly, in some embodiments, the method includes identifying hidden clinical labels in ECG images that are associated with a disease. Additionally, or alternatively, in some embodiments, the methods disclosed herein detect underlying cardiovascular disorders and/or predict their future risk.

In some embodiments, the methods disclosed herein include monitoring patients previously diagnosed with a cardiac disease and/or detecting a further cardiac condition in such patients. For example, in some embodiments, the methods disclosed herein include monitoring and/or detecting conditions in patients with hypertrophic cardiomyopathy (HCM), a genetic disease that is associated with increased risk of atrial fibrillation, stroke, and sudden cardiac death. In some embodiments, the condition is left ventricular (LV) systolic dysfunction. In such embodiments, the method includes training a machine-learning algorithm to detect LV systolic dysfunction in HCM patients according to one or more of the embodiments disclosed herein. For example, the training of the nodes may include creating a series of image-based datasets (e.g., normal subset and diseased subset) from HCM patients with any one or more ECG lead layouts, optionally pre-training the nodes on a pre-defined set of labels, and then training the nodes on the image-based dataset to detect features of LV systolic dysfunction among HCM patients. The image-based dataset may include any one or more ECG formats according to the embodiments disclosed herein (e.g., 12-lead ECG signal data in various formats/frequencies from any one or more sources). Following such training, the algorithm forms a superhuman reader of ECG images and photos in any layout. In some embodiments, the trained algorithm recognizes individual leads of the ECG regardless of their location on the page, detects hidden features of LV systolic dysfunctions amongst HCM patients that are not discernable to humans, or a combination thereof. In some embodiments, the articles and methods disclosed herein facilitate decentralized tracking of systolic function amongst patients with HCM.

Without wishing to be bound by theory, it is believed that the methods disclosed herein represent the first application of artificial intelligence on ECG images regardless of their printed format. As opposed to existing methods, which rely on raw waveform data, the methods disclosed herein are capable of diagnosing the ECGs as a super-human reader, identifying both the location of leads (like human readers) as well as the hidden signatures of disease (that humans cannot see). Therefore, the methods disclosed herein can identify clinical and hidden diagnoses from images and photographs of ECG taken from any commonly available and easily accessible real-world printed or digital ECG image layout. Accordingly, the methods disclosed herein provide a new option for most healthcare settings that have not been optimized for storing and processing signal data in real-time and rely on printed or scanned ECG systems. Additionally, in some embodiments, the methods disclosed herein are automated, such that human input is not required for data extraction. Furthermore, by utilizing printed images, the method disclosed herein allow for better real-time feedback to clinicians on what portions of the ECG were used by the model to ascribe a certain hidden label, allowing for contextualization that can aid in their acceptance in clinical workflow.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

### Introduction

Left ventricular (LV) systolic dysfunction is associated with over 8-fold increased risk of subsequent heart failure and nearly 2-fold risk of premature death. While early diagnosis can effectively lower this risk, individuals are often diagnosed after developing symptomatic disease due to lack of effective screening strategies. The diagnosis traditionally relies on echocardiography, a specialized imaging modality that is resource intensive to deploy at scale. Algorithms using raw signals from electrocardiography (ECG) have been developed as a strategy to detect LV systolic dysfunction. However, clinicians, particularly in remote settings, do not have access to ECG signals. The lack of interoperability in signal storage formats from ECG devices further limits the broad uptake of such signal-based models. The use of ECG images is an opportunity to implement interoperable screening strategies for LV systolic dysfunction.

We previously developed a deep learning approach of format-independent inference from real-world ECG images (Sangha V, Mortazavi B J, Haimovich A D, Ribeiro A H, Brandt C A, Jacoby D L, Schulz W L, Krumholz H M, Ribeiro A L P, Khera R. Automated multilabel diagnosis on electrocardiographic images and signals. Nat Commun. 2022; 13:1583). The approach can interpretably diagnose cardiac conduction and rhythm disorders using any layout of real-world 12-lead ECG images and can be accessed on web- or application-based platforms. Extension of this artificial intelligence (AI)-driven approach to ECG images to screen for LV systolic dysfunction could rapidly broaden access to a low-cost, easily accessible, and scalable diagnostic approach to underdiagnosed and undertreated at-risk populations. This approach adapts deep learning for end-users without disruption of data pipelines or clinical workflow. Moreover, the ability to add localization of predictive cues in the ECG images relevant to the LV can improve the uptake of these models in clinical practice.

In this study, we present a model for accurate identification of LV ejection fraction (LVEF) less than 40%, a threshold with therapeutic implications, based on ECG images. We developed, tested, and externally validated this approach using paired ECG-echocardiographic data from large academic hospitals, rural hospital systems, and a prospective cohort study.

### Methods

### Data Source for Model Development

We used 12-lead ECG signal waveform data from the Yale New Haven Hospital (YNHH) collected between 2015 and 2021. These ECGs were recorded as standard 12-lead recordings sampled at a frequency of 500 Hz for 10 seconds. These were recorded on multiple different machines and a majority were collected using Philips PageWriter machines and GE MAC machines. Among patients with an ECG, those with a corresponding transthoracic echocardiogram (TTE) within 15 days of obtaining the ECG were identified from the YNHH electronic health records. LVEF values were extracted based on a cardiologist's read of the nearest TTE to each ECG. To augment the evaluation of models built on an image dataset generated from this YNHH signal waveform, six sets of ECG image datasets were used for external validation.

### Data Preprocessing

All ECGs were analyzed to determine whether they had 10 seconds of continuous recordings across all 12 leads. The 10-second samples were preprocessed with a one-second median filter, subtracted from the original waveform to remove baseline drift in each lead, representing processing steps pursued by ECG machines before generating printed output from collected waveform data.

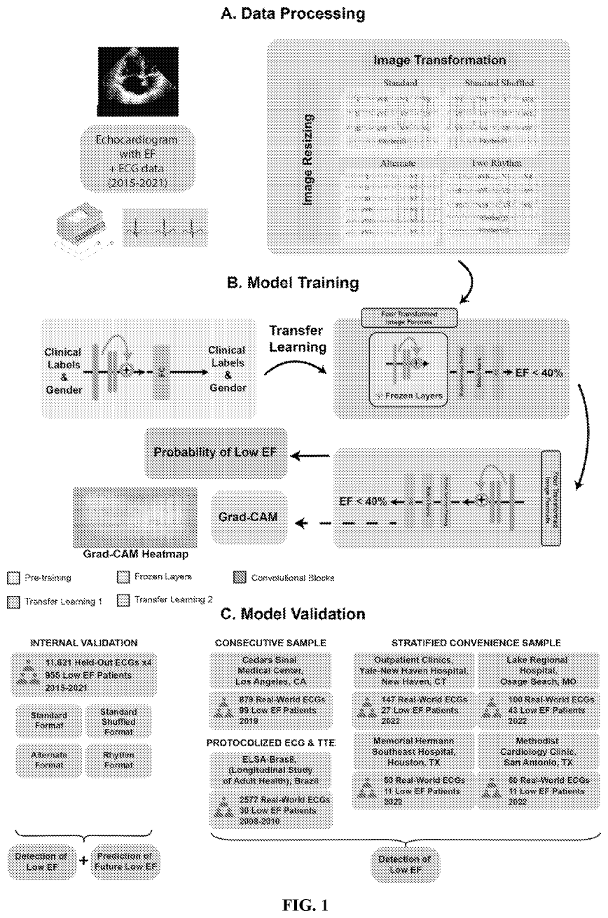

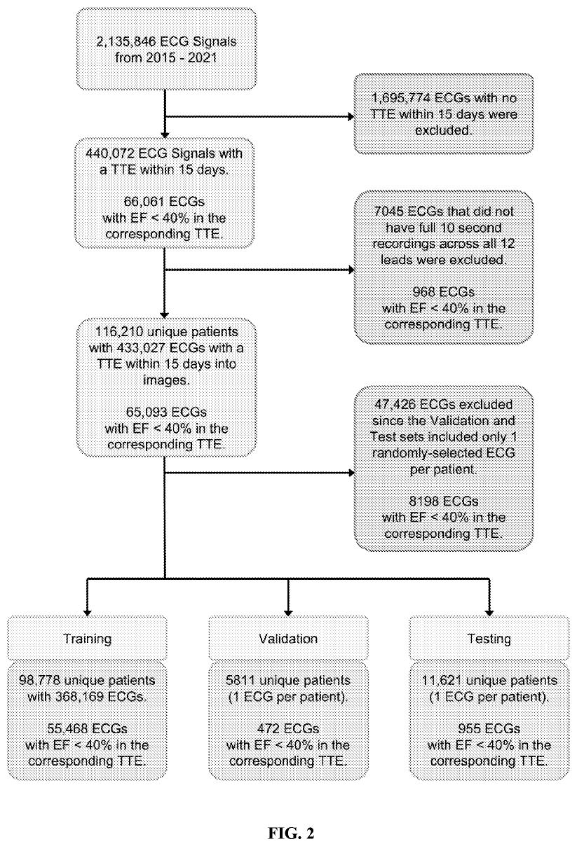

ECG signals were transformed into ECG images using the Python library ecg-plot (ECG Plot Python Library. Accessed at https://pypi.org/project/ecg-plot/on May 25, 2022), and stored at 100 DPI. Images were generated with a calibration of 10 mm/mV, which is standard for printed ECGs in most real-world settings. In sensitivity analyses, we evaluated model performance on images calibrated at 5 and 20 mm/mV. All images, including those in train, validation, and test sets, were converted to greyscale, followed by down-sampling to 300×300 pixels regardless of their original resolution using Python Image Library (PIL v9.2.0). To ensure that the model was adaptable to real-world images, which may vary in formats and the layout of leads, we created a dataset with different plotting schemes for each signal waveform recording (FIG. 1). This strategy has been used to train a format-independent image-based model for detecting conduction and rhythm disorders as well as the hidden label of gender. The model in this study learned ECG lead-specific information based on the label regardless of the location of the lead.

Four formats of images were included in the training image dataset (FIG. 1). The first format was based on the standard printed ECG format in the United States, with four 2.5-second columns printed sequentially on the page. Each column contained 2.5-second intervals from three leads. The full 10-second recording of the lead I signal was included as the rhythm strip. The second format, a two-rhythm format, added lead II as an additional rhythm strip to the standard format. The third layout was the alternate format which consisted of two columns, the first with six simultaneous 5-second recordings from the limb leads, and the second with six simultaneous 5-second recordings from the precordial leads, without a corresponding rhythm lead. The fourth format was a shuffled format, which had precordial leads in the first two columns and limb leads in the third and fourth. All images were rotated a random amount between −10 and 10 degrees before being input into the model to mimic variations seen in uploaded ECGs and to aid in prevention of overfitting.

The process of converting ECG signals to images was independent of model development, ensuring that the model did not learn any aspects of the processing that generated images from the signals. All ECGs were converted to images in all different formats without conditioning on clinical labels. The validation required uploaded images to be upright, cropped to the waveform region, with no brightness and contrast consideration as long as the waveform is distinguishable from the background and lead labels are discernible.

### Experimental Design

Each included ECG had a corresponding LVEF value from its nearest TTE within 15 days of recording. Low LVEF was defined as LVEF<40%, the cutoff used as an indication for most guideline-directed pharmacotherapy for heart failure (Heidenreich P A, Bozkurt B, Aguilar D, Allen L A, Byun J J, Colvin M M, Deswal A, Drazner M H, Dunlay S M, Evers L R, Fang J C, Fedson S E, Fonarow G C, Hayek S S, Hernandez A F, Khazanie P, Kittleson M M, Lee C S, Link M S, Milano C A, Nnacheta L C, Sandhu A T, Stevenson L W, Vardeny O, Vest A R, Yancy C W. 2022 AHA/ACC/HFSA Guideline for the Management of Heart Failure: A Report of the American College of Cardiology/American Heart Association Joint Committee on Clinical Practice Guidelines. Circulation. 2022; 101161CIR0000000000001063). Patients with at least one ECG within 15 days of its nearest TTE were randomly split into training, validation, and held-out test patient level sets (85%, 5%, 10%, FIG. 2). This sampling was stratified by whether a patient had ever had LVEF<40% to ensure cases of preserved and reduced LVEF were split proportionally among the sets. In the training cohort, all ECGs within 15 days of a TTE were included for all patients to maximize the data available. In validation and testing cohorts, only one ECG was included per patient to ensure independence of observations in the assessment of performance metrics. This ECG was randomly chosen amongst all ECGs within 15 days of a TTE. Additionally, to ensure that model learning was not affected by the relatively lower frequency of LVEF<40%, higher weights were given to these cases at the training stage based on the effective number of samples class sampling scheme.

### Model Training

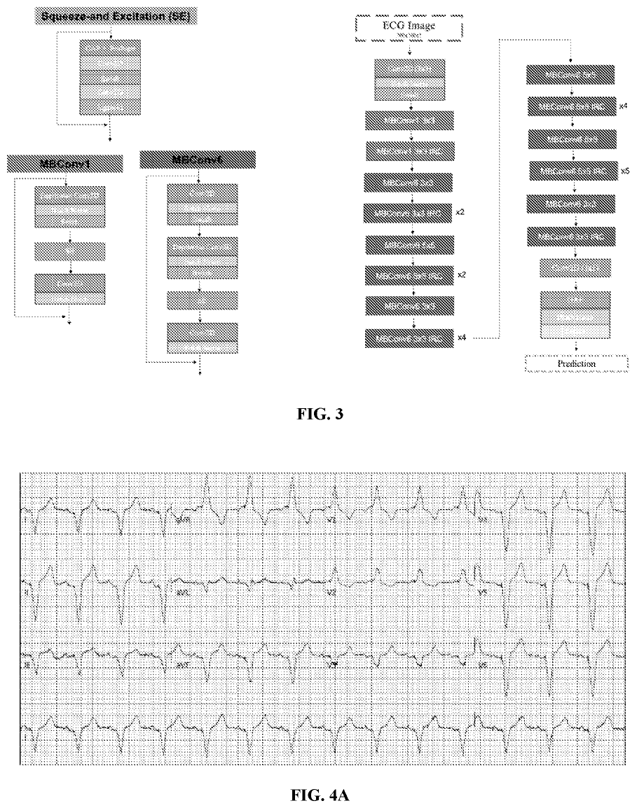

We built a convolutional neural network model based on the EfficientNet-B3 architecture (Mingxing Tan and Quoc V Le. EfficientNet: Rethinking model scaling for convolutional neural networks. International Conference on Machine Learning, 2019), which previously demonstrated an ability to learn and identify both rhythm and conduction disorders, as well as the hidden label of gender in real-world ECG images (Sangha V, Mortazavi B J, Haimovich A D, Ribeiro A H, Brandt C A, Jacoby D L, Schulz W L, Krumholz H M, Ribeiro A L P, Khera R. Automated multilabel diagnosis on electrocardiographic images and signals. Nat Commun. 2022; 13:1583). The EfficientNet-B3 model requires images to be sampled at 300×300 square pixels, includes 384 layers, and has over 10 million trainable parameters (FIG. 3). We utilized transfer learning by initializing model weights as the pretrained EfficientNet-B3 weights used to predict the six physician-defined clinical labels and gender from Sangha et al. Other than the weights, clinical and gender labels were not input to the current model. We first only unfroze the last four layers and trained the model with a learning rate of 0.01 for 2 epochs, and then unfroze all layers and trained with a learning rate of 5×10⁻⁶ for 6 epochs. We used an Adam optimizer, gradient clipping, and a minibatch size of 64 throughout training. The optimizer and learning rates were chosen after hyperparameter optimization. For both stages of training the model, we stopped training when validation loss did not improve in 3 consecutive epochs.

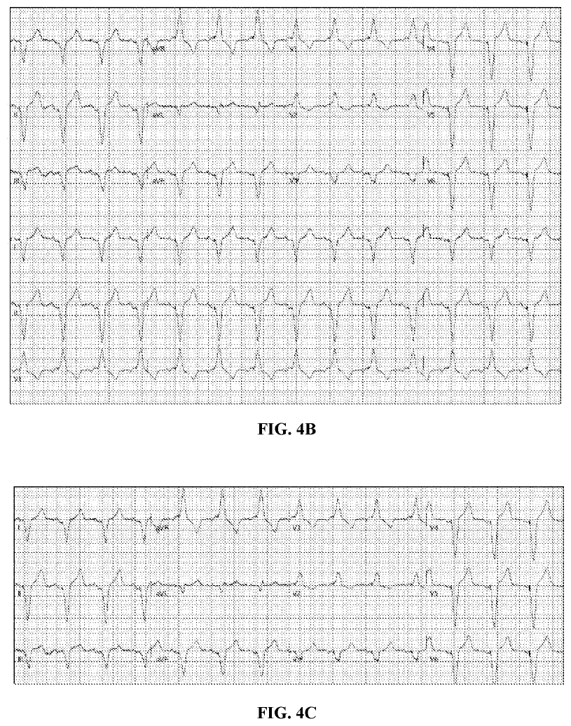

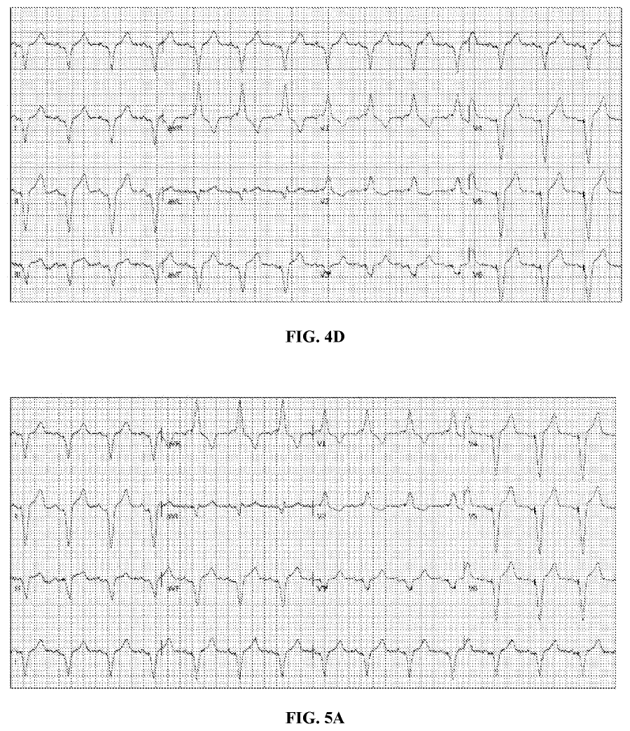

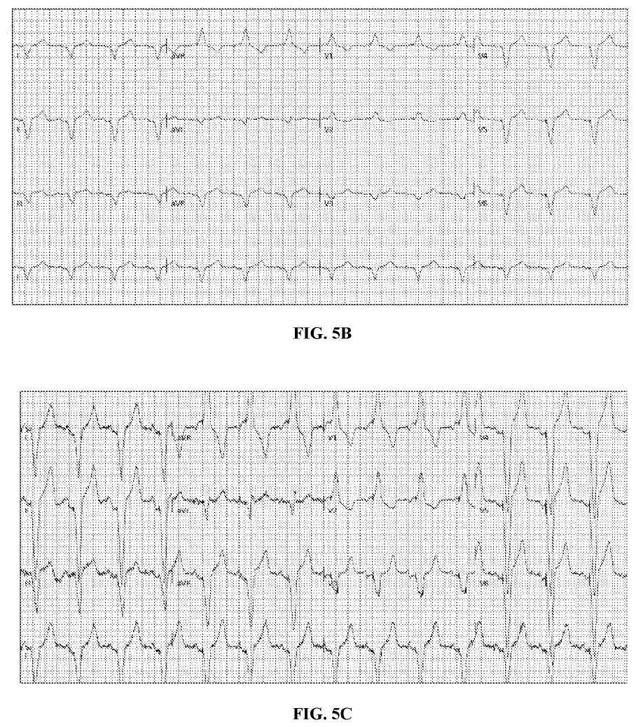

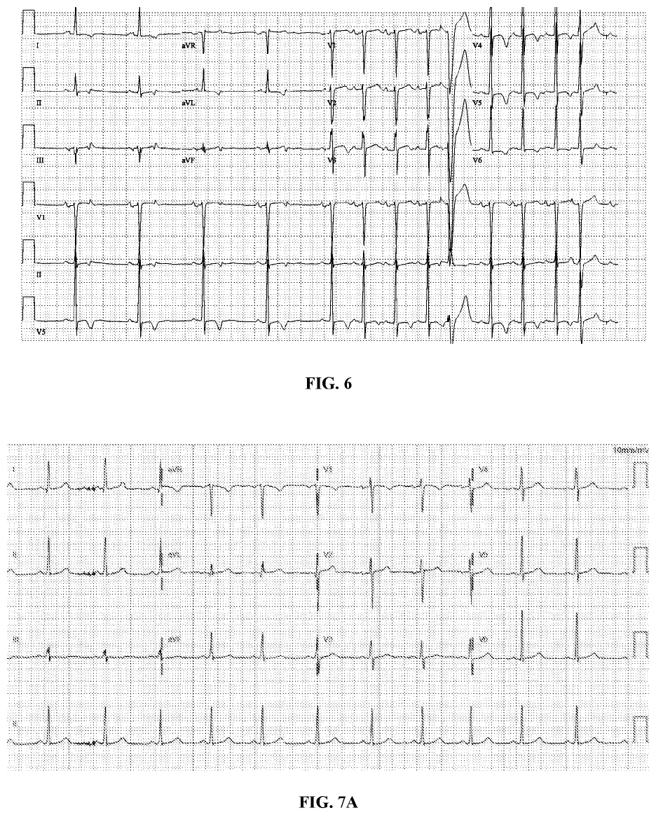

We trained and validated our model on a generated image dataset that had equal numbers of standard, two-rhythm, alternate, and standard shuffled images (FIG. 1). In sensitivity analyses, the model was validated on three novel ECG layouts constructed from the held-out set to assess its performance on ECG formats not encountered in the training process. These novel ECG outlines included three-rhythm (with leads I, II, and V1 as the rhythm strip), no rhythm, and rhythm on top formats (with lead I as the rhythm strip located above the 12-lead, FIGS. 4A-D). Additional sensitivity analyses were performed using ECG images calibrated at 5, 10, and 20 mm/mV (FIGS. 5A-C). A custom class-balanced loss function (weighted binary cross-entropy) based on the effective number of samples was used given the lower frequency of the LVEF<40% label relative to those with an LVEF≥40%. Furthermore, model performance was evaluated in a 5-fold cross validation analysis using the original derivation (train and validation) set. A patient-level split stratified by LVEF<40% vs ≥40% was pursued in this analysis and model performance was assessed on the held-out test set.

### External Validation

We pursued a series of validation studies. These represented both clinical and population-based cohort studies. Clinical validation represented non-synthetic image datasets from clinical settings spanning (1) consecutive patients undergoing outpatient echocardiography at the Cedars Sinai Medical Center in Los Angeles, CA, and (2) stratified convenience samples of LV systolic dysfunction and non-LV systolic dysfunction ECGs from four different settings (a) outpatient clinics of YNHH, (b) inpatient admissions at Lake Regional Hospital (LRH) in Osage Beach, MO, (c) inpatient admissions at Memorial Hermann Southeast Hospital in Houston, TX, (d) outpatient visits and inpatient admissions at Methodist Cardiology Clinic in San Antonio, TX. In addition, we validated our approach in the prospective cohort from Brazil, the Brazilian Longitudinal Study of Adult Health (ELSA-Brasil), with protocolized ECG and echocardiogram in study participants.

Inclusion and exclusion criteria for external validation sets were similar to the internal YNHH dataset. Patients were limited to those having a 12-lead ECG within 15 days of a TTE with reported LVEF. For patients with more than one TTE in this interval, the LVEF from the nearest TTE was used for analysis.

At Cedars Sinai, all index ECG images from consecutive patients undergoing outpatient visits during January through March 2019, representing 879 individuals, including 99 with LVEF<40%, were included. These analyses were performed in a fully federated and blinded fashion without access to the ECGs by the algorithm's developers.

For the other clinical validation sites, a stratified convenience sample enriched for low LVEF was drawn. This was done to evaluate the broad use in a clinical setting by practicing clinicians without access to a research dataset. Our preliminary assessment of LV systolic dysfunction prevalence in outpatient and inpatient settings were 10% and 20%, respectively. We sought to achieve twice this prevalence in our external validation data in these sites to ensure our performance was not driven by patients with preserved LVEF and that the model could detect those with LV systolic dysfunction. Specifically, a 1:4 ratio of ECGs corresponding to LVEF<40% and ≥40% was sought at three of the four sites (YNHH, Memorial Hermann Southeast Hospital, and Methodist Cardiology Clinic). At the fourth site, LRH, a 1:2 ratio was requested to better measure the model's discriminative ability in an inpatient-only setting.

In addition to the clinical validation studies, where concurrent ECG and echocardiogram are always clinically indicated, imposing a selection of the population, we evaluated our model in the ELSA-Brasil study, a community-based prospective cohort in Brazil that obtained ECG and echocardiography from participants on the enrollment visit between 2008-2010. This set included data from 2,577 individuals, including 30 from individuals with LVEF<40%.

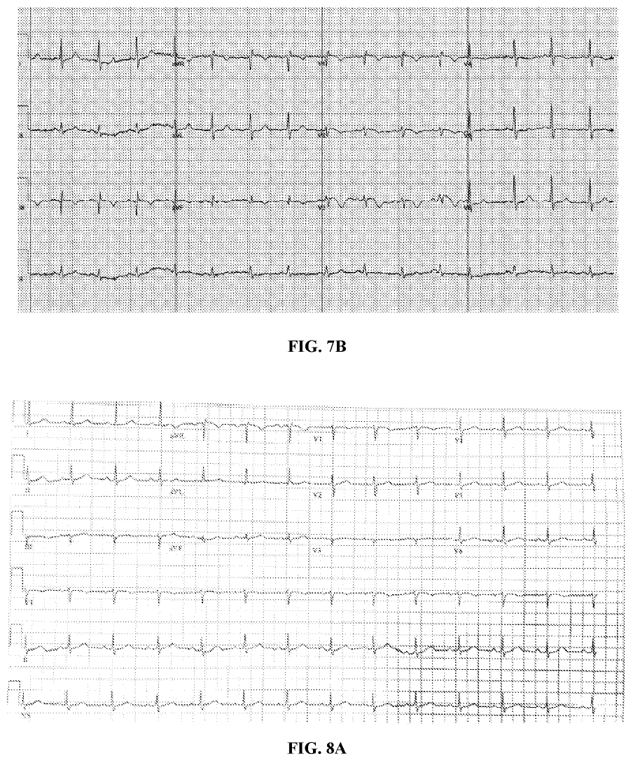

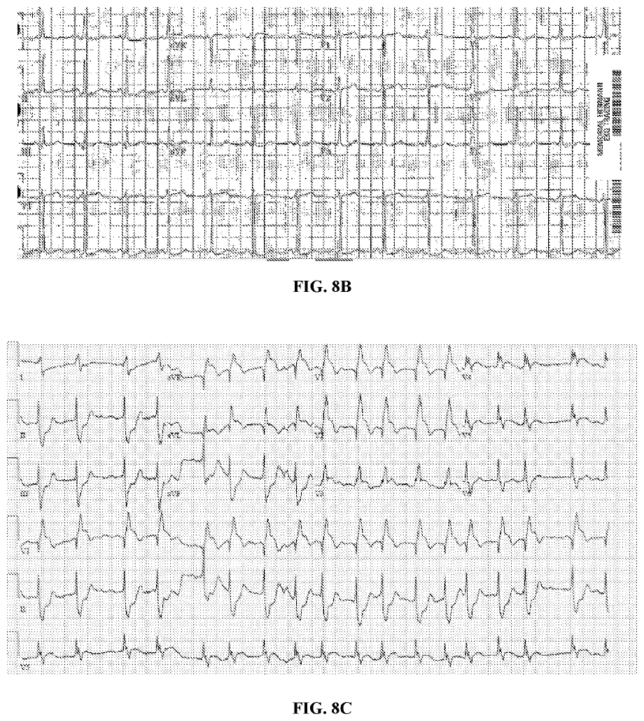

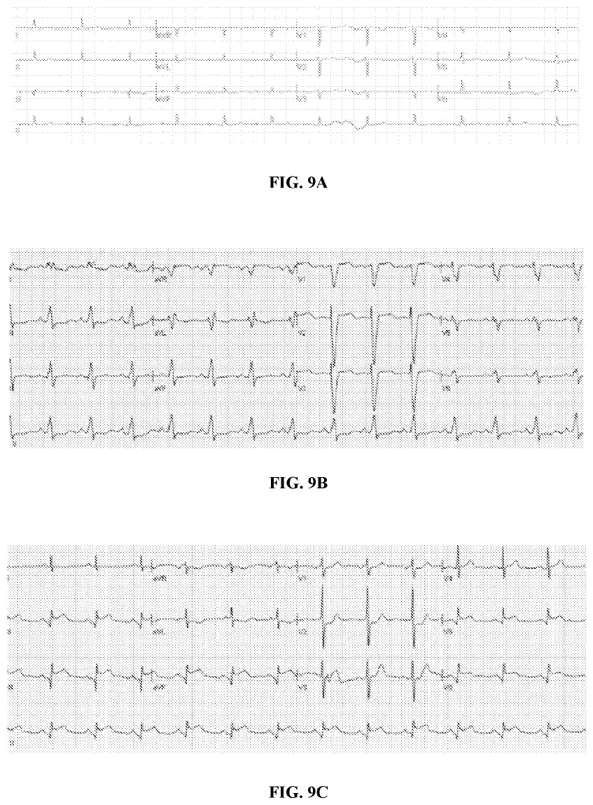

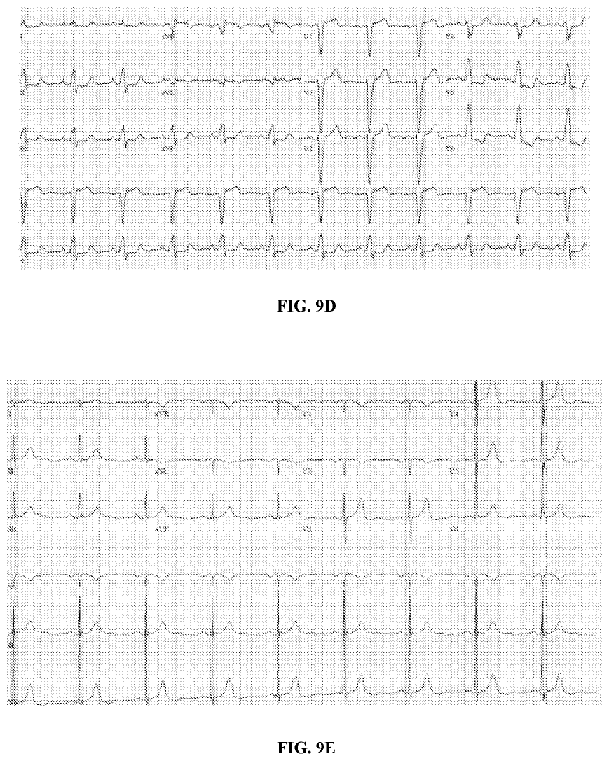

Before validation, patient identifiers, ECG measurements, and reported diagnoses were removed from all ECG images. The differences in ECG layouts and the procedures for validation are described in further detail in the Online Supplement. Deidentified samples of ECG images are presented in FIG. 6 (Cedars Sinai Medical Center), FIGS. 7A-B (YNHH and LRH), FIGS. 8A-C (Memorial Hermann Southeast Hospital), and FIGS. 9A-E (Methodist Cardiology Clinic), and images are available from the authors upon request.

### Localization of Model Predictive Cues

We used Gradient-weighted Class Activation Mapping (Grad-CAM) to highlight which portions of an image were important for predicting LVEF<40% (Selvaraju R R, Cogswell M, Das A, Vedantam R, Parikh D, Batra D. Grad-CAM: Visual Explanations from Deep Networks via Gradient-based Localization. 2017 Ieee International Conference on Computer Vision (Iccv). 2017; 618-626). We calculated the gradients on the final stack of filters in our EfficientNet-B3 model for each prediction and performed a global average pooling of the gradients in each filter, emphasizing those that contributed to a prediction. We then multiplied these filters by their importance weights and combined them across filters to generate Grad-CAM heatmaps. We averaged class activation maps among 100 positive cases with the most confident model predictions for LVEF<40% across ECG formats to determine the most important image areas for the prediction of low LVEF. We took an arithmetic mean across the heatmaps for a given image format and overlayed this average heatmap across a representative ECG before conversion of the image to grayscale. The Grad-CAM intensities were converted from their original scale (0-1) to a color range using the jet colormap array in the Python library matplotlib. This colormap was then overlaid on the original ECG image with an alpha of 0.3. The activation map, a 10×10 array was upsampled to the original image size using the bilinear interpolation built into TensorFlow v2.8.0. We also evaluated the Grad-CAM for individual ECGs to evaluate the consistency of the information on individual examples.

### Preprocessing Strategies for Noisy Input Data

Standard input requirements for our image-based model include ECG images limited to 12-lead tracings with an upright orientation, minimal rotation, solid background, and no peripheral annotations. To mitigate the impact of noisy input data on model predictions in real-world applications, we built in an automated preprocessing function that includes two major steps: (1) Straightening and cropping: In this step, the input ECG image is automatically straightened to correct for rotations and then cropped to remove the peripheral elements. The output of this preprocessing step is a 12-lead tracing without surrounding annotations and patient identifiers. (2) Quality evaluation and standardization: The algorithm computes the mean pixel-level brightness and contrast values for input images and evaluates them against the brightness and contrast of images used in model development. The brightness and contrast are either scaled to the mean values of the development population before predictions. For ECGs with extreme deviations of brightness and contrast (50% above or below the development set) are flagged to be out-of-range so a better-quality image can be acquired and input.

We evaluated the model calibration across the variations of photo brightness and contrast. For this analysis, we used the Python Image Library (PIL) to adjust the input image qualities. A total of 200 ECGs were randomly selected from the held-out test set in a 1:4 ratio for LVEF<40% and ≥40%, respectively. Variations of the original image were generated with brightness and contrast between 0.5 to 1.5 times the original values and were used in this sensitivity analysis.

### Statistical Analysis

Categorical variables were presented as frequency and percentages, and continuous variables as means and standard deviations or median and interquartile range, as appropriate. Model performance was evaluated in the held-out test set and external ECG image datasets. We used area under the receiver operator characteristic (AUROC) to measure model discrimination. The cut-off for binary prediction of LV systolic dysfunction was set at 0.10 for all internal and external validations, based on the threshold that achieved a sensitivity of over 90% in the internal validation set. We also assessed the area under the precision-recall curve (AUPRC), sensitivity, specificity, positive predictive value (PPV), negative predictive value (NPV), and diagnostic odds ratio. 95% CIs for AUROC and AUPRC were calculated using DeLong's algorithm and bootstrapping with 1000 variations for each estimate, respectively (DeLong E R, DeLong D M, Clarke-Pearson D L. Comparing the areas under two or more correlated receiver operating characteristic curves: a nonparametric approach. Biometrics. 1988; 44:837-845; Sun X, Xu W. Fast implementation of DeLong's algorithm for comparing the areas under correlated receiver operating characteristic curves. IEEE Signal Process Lett. 2014; 21:1389-1393). Model performance was assessed across demographic subgroups and ECG outlines, as described above. We conducted further sensitivity analyses of model performance across ECG calibrations. We also evaluated model performance across PR intervals (>200 ms vs. ≤200 ms) and after excluding ECGs with paced rhythms, conduction disorders, atrial fibrillation, and atrial flutter. Moreover, we assessed the association of the model's predicted probability of LV systolic dysfunction across LVEF categories.

Next, we evaluated the future development of LV systolic dysfunction in time-to-event models using a Cox proportional hazards model. In this analysis, we took the first temporal ECG from the patients in the held-out test set and then modeled the first development of LVEF<40% across the groups of patients who screened positive but did not have concurrent LV systolic dysfunction (false positives), and those that screened negative (true negative) from this first ECG, with censored at death or end of study period in June 2021. Additionally, we computed an adjusted hazard ratio that accounted for differences in age, sex, and baseline LVEF at the time of index screening for visualization of survival trends. Analytic packages used in model development and statistical analysis are reported in Table 1. All model development and statistical analyses were performed using Python 3.9.5 and the level of significance was set at an alpha of 0.05.

TABLE 1
Analytic packages and language used for model
development and statistical analysis

| Programming Language/Package | Version |
|---|---|
| Python | 3.9.5 |
| TensorFlow | 2.8.0 |
| scikit-learn | 0.24.2 |
| pandas | 1.3.1 |
| numpy | 1.19.5 |

### External Validation Data and Procedures

The model was externally validated on ECG images obtained through three separate sampling strategies:
1—Consecutive inclusion at Cedars Sinai Medical Center in Los Angeles, CA, USA.
2—Stratified convenience sampling at 4 centers, evaluating the logistics of deploying the model directly by clinicians:
   a. Outpatient clinics of Yale New Haven Hospital (YNH) across the state of Connecticut (CT), USA,
   b. Lake Regional Hospital (LRH) at Osage Beach, MO, USA,
   c. Memorial Hermann Southeast Hospital at Houston, TX, USA, and
   d. Methodist Cardiology Clinic at San Antonio, TX, USA.
3—Community-based prospective sampling in Brazilian Longitudinal Study of Adult Health (ELSA-Brasil)
   The sampling strategies and ECG image acquisition details are described for each center below.

Cedars Sinai Medical Center: ECG images were obtained during outpatient encounters of patients at Cedars Sinai Medical Center between January through March 2019. A total of 879 ECGs from unique individuals, including 99 with LVEF<40%, were included in this set. Clinically used PDFs of ECGs were collected for model validation. These ECGs had three rhythm strips at the bottom (V1, II, and V5), which was different from the original four layouts included in the model training. This sample represents all individuals who underwent an echocardiography at Cedars Sinai Medical Center during this period. The prevalence of LV systolic dysfunction in this sample was not pre-specified, hence represents the true prevalence rate of individuals with low LVEF in this population in this interval.

Outpatient Clinics of YNHH: ECG images from outpatient clinics of YNHH were obtained during January through March 2022 and included 147 ECGs from unique individuals, 27 with LVEF<40%. This was a convenience sample, with oversampling individuals with LVEF<40% to achieve a target prevalence of 20% for LV systolic dysfunction, which was estimated to be twice as large as the underlying prevalence of LV systolic dysfunction in this population (10%). The ECG images were manually captured through image capture from electronic health record. These images had a similar layout to the standard ECG format used in model training but had the lead II rather than lead I as the rhythm strip. Moreover, there were several real-world noise artifacts in these images, including the shade of the page, vertical lines demarcating the leads, and differences in the location of the lead labels.

Lake Regional Hospital (LRH): LRH is a community hospital and part of a rural US hospital system in Osage Beach, MO. Data from this external set included 100 ECG images, with 43 from patients with LVEF<40%. Individuals with LVEF<40% were oversampled in to achieve a target prevalence of 40% for LV systolic dysfunction in this convenience sample. The ECG images in this sample had a similar layout as the standard ECG format in the train set but had lead II rather than lead I as the rhythm strip. The images were obtained through image captured from the electronic health records of individuals. There were unique noise real-world artifacts present in these images too, including a different background color, the layout of the grid over which the waveform data are displayed, as well as the location and the font of the lead label.

Memorial Hermann Southeast Hospital: 50 ECG images were obtained from inpatient admissions at Memorial Hermann Southeast Hospital in Houston, TX. Patients with LV systolic dysfunction were oversampled for a target prevalence of 20% in this convenience sample, which included 11 individuals with LVEF<40% in the final sample. ECGs in this sample were in printed format and had three rhythm leads (V1, II, and V5) at the bottom The ECG paper copies in the medical records were scanned.

Methodist Cardiology Clinic: This dataset included ECGs from 50 individuals, including 11 individuals with LVEF<40% from inpatient admissions or outpatient visits at Methodist Cardiology Clinic in San Antonio, TX. Individuals with LVEF<40% were oversampled. ECGs were obtained through screenshots of electronic medical records and had several different outlines, including one (lead II), two (leads II and V1), or three (leads II, V1, or V5) leads as the rhythm strips at the bottom.

ELSA-Brasil: The Brazilian Longitudinal Study of Adult Health (ELSA-Brasil) studied the development and progression of clinical and subclinical chronic diseases, particularly cardiovascular diseases, and diabetes. The study enrolled 15,105 participants from the community at 6 academic centers in Brazil between 2008-2019. All active or retired employees of the six institutions, aged between 35 and 74 years, were eligible for the study. The participants underwent interview, physical examination, and laboratory testing at baseline (2008-2010). This was followed by annual telephone surveillance for incident events and behavioral risk factors and quadrennial face-to-face interviews and examinations. In this prospective study, echocardiography and ECG data were obtained from enrolled participants by protocol and not by indication. A total of 2,577 individuals, including 30 with LVEF<40%, had ECG-echocardiography data, all of whom were included in this validation set. Notably, the prevalence of LV systolic dysfunction in this sample is lower than the other external sets (˜1%).

### Results

### Study Population

Out of the 2,135,846 ECGs obtained between 2015 to 2021, 440,072 were from patients who had TTEs within 15 days of obtaining the ECG. Overall, 433,027 had a complete ECG recording, representing 10 seconds of continuous recordings across all 12 leads. These ECGs were drawn from 116,210 unique patients and were split into train, validation, and test sets at a patient level (FIG. 2).

A total of 116,210 individuals with 385,601 ECGs constituted the study population, representing those included in the training, validation, and test sets. Individuals in the model development population had a median age of 68 years (IQR 56, 78) at the time of ECG recording, and 59,282 (51.0%) were women. Overall, 75,928 (65.3%) were non-Hispanic white, 14,000 (12.0%) non-Hispanic Black, 9,349 (8.0%) Hispanic, and 16,843 (14.5%) were from other races. A total of 56,895 (14.8%) ECGs had a corresponding echocardiogram with an LVEF below 40%, 36,669 (9.5%) had an LVEF greater than or equal to 40% but less than 50%, and 292,037 (75.7%) had LVEF 50% or greater (Table 2).

TABLE 2
Baseline characteristics of study population. Data presented as median
[IQR] for age and number (percent) for other variables.

| | | | ECGs with | ECGs with |
|---|---|---|---|---|
| | | | Corresponding | Corresponding |
| Characteristic | Patients | ECGs | TTE with EF <40% | TTE with EF ≥40% |
| Number | 116,210 | 385,601 | 56,895 | 328,706 |

Sex

| | | | | | | | | |
|---|---|---|---|---|---|---|---|---|
| Female | 59,282 | (51.0%) | 175,563 | (45.5%) | 18,867 | (33.2%) | 156,696 | (47.7%) |
| Male | 56,917 | (49.0%) | 210,006 | (54.5%) | 38,015 | (66.8%) | 171,991 | (52.3%) |
| Missing | 11 | (0.0%) | 22 | (0.0%) | 13 | (0.0%) | 9 | (0.0%) |
| Age (years) | 68 | [56-78] | 68 | [57-78] | 70 | [59-79] | 68 | [57-78] |

Race

| | | | | | | | | |
|---|---|---|---|---|---|---|---|---|
| Hispanic | 9,349 | (8.0%) | 32,794 | (8.5%) | 4,844 | (8.5%) | 27,950 | (8.5%) |
| White | 75,928 | (65.3%) | 246,135 | (63.8%) | 35,695 | (62.7%) | 210,440 | (64.0%) |
| Black | 14,000 | (12.0%) | 54,393 | (14.1%) | 9,502 | (16.7%) | 44,891 | (13.7%) |
| Other | 16,843 | (14.5%) | 52,279 | (13.6%) | 6,854 | (12.0%) | 45,425 | (13.8%) |

ECG Abnormalities

| | | | | | | | |
|---|---|---|---|---|---|---|---|
| A-Fib or Flutter | — | 61,073 | (15.8%) | 11,903 | (20.9%) | 49,170 | (15.0%) |
| Paced ECGs | — | 22,324 | (5.8%) | 7,920 | (13.9%) | 14,404 | (4.4%) |
| PR Interval >200 ms | — | 46,886 | (12.2%) | 8,128 | (14.3%) | 38,758 | (11.8%) |
| LBBB | — | 18,807 | (4.9%) | 7,141 | (12.6%) | 11,666 | (3.5%) |
| RBBB | — | 34,318 | (8.9%) | 5,847 | (10.3%) | 28,471 | (8.7%) |

Abbreviations: ECGs, electrocardiograms; EF, ejection fraction; A-Fib, atrial fibrillation; LBBB, left bundle branch block; RBBB, right bundle branch block.

### Detection of LV Systolic Dysfunction

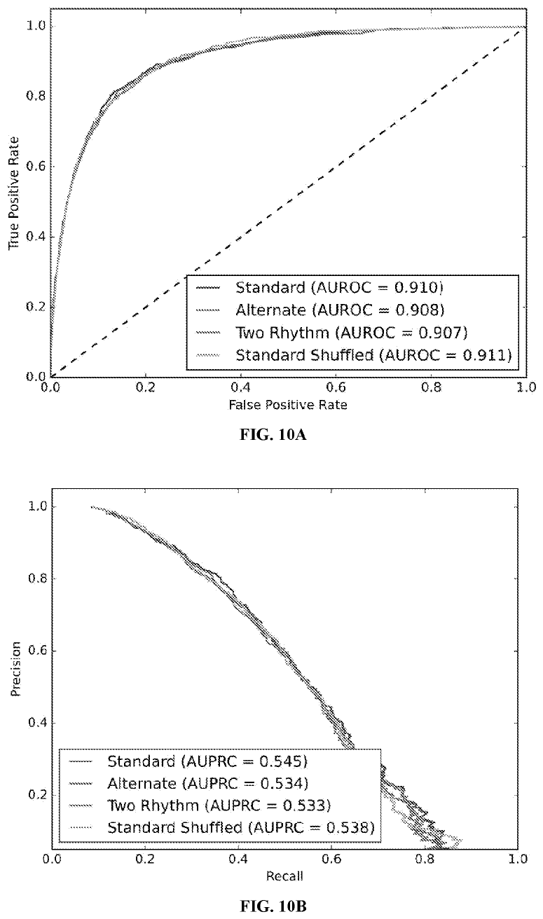

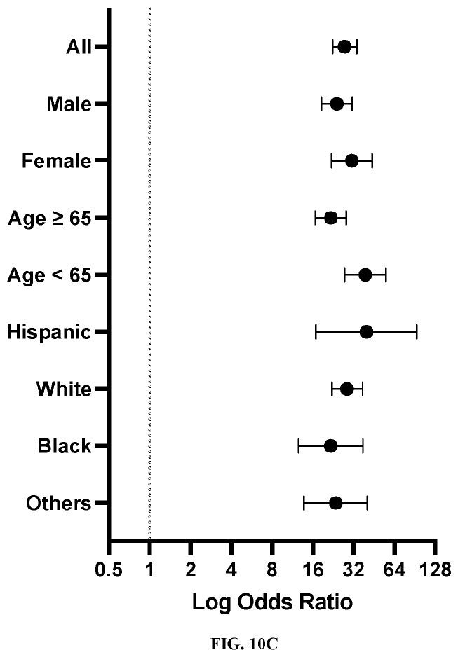

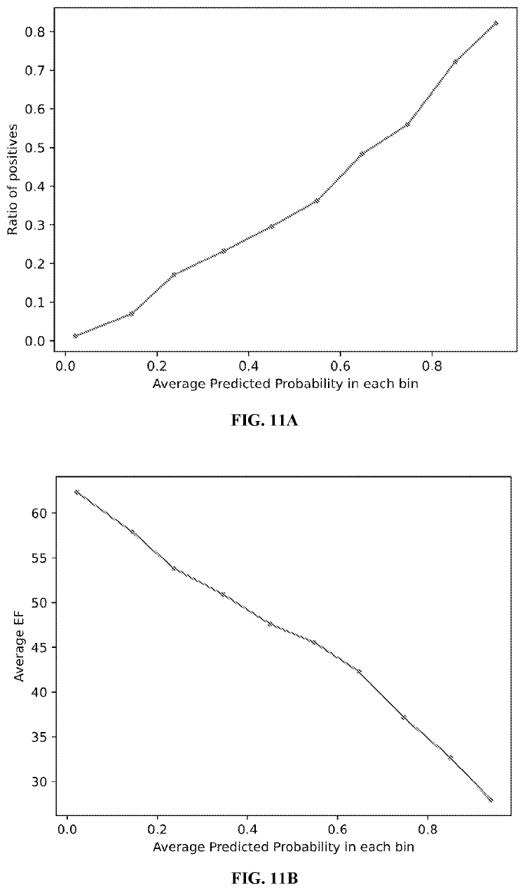

The model's AUROC for detecting LVEF<40% on the held-out test set composed of standard images was 0.91 and its AUPRC was 0.55 (FIGS. 10A-C). A probability threshold for predicting LVEF<40% was chosen based on a sensitivity of 0.90 or higher in the validation subset, with a specificity of 0.77 at this threshold in the internal validation set. With this threshold, the model had sensitivity and specificity of 0.89 and 0.77 in the held-out test set and PPV and NPV of 0.26 and 0.99, respectively. Overall, an ECG suggestive of LV systolic dysfunction portended over 27-fold higher odds (OR 27.5, 95% CI, 22.3-33.9) of LV systolic dysfunction on TTE (Table 3). The model's performance was comparable across subgroups of age, sex, and race (Table 3 and FIGS. 10A-C). In a cross-validation analysis, model performance remained consistent across 5 splits and was similar to the performance of the original model (Table 4). Moreover, across successive deciles of the model predicted probabilities, the proportion of individuals with LV systolic dysfunction increased while the mean LVEF decreased (FIGS. 11A-B).

TABLE 3
Performance of model on test images across demographic subgroups in the held-out test set.

| Labels | Number | PPV | NPV | Specificity | Sensitivity | AUROC | AUPRC | F1 Score |
|---|---|---|---|---|---|---|---|---|
| All | 11621 | 0.257 | 0.988 | 0.769 | 0.892 | 0.910 | 0.545 | 0.399 |
| | (100%) | | | | | (0.901-0.919) | (0.511-0.579) | |
| Male | 5952 | 0.285 | 0.984 | 0.735 | 0.897 | 0.901 | 0.583 | 0.433 |
| | (51.2%) | | | | | (0.889-0.914) | (0.539-0.621) | |
| Female | 5668 | 0.215 | 0.991 | 0.802 | 0.884 | 0.917 | 0.470 | 0.346 |
| | (48.8%) | | | | | (0.903-0.932) | (0.416-0.530) | |
| ≥65 | 6550 | 0.252 | 0.985 | 0.717 | 0.896 | 0.892 | 0.522 | 0.393 |
| | (56.4%) | | | | | (0.880-0.905) | (0.480-0.561) | |
| <65 | 5068 | 0.266 | 0.991 | 0.833 | 0.886 | 0.931 | 0.590 | 0.410 |
| | (43.6%) | | | | | (0.916-0.945) | (0.534-0.655) | |
| Hispanic | 942 | 0.253 | 0.992 | 0.802 | 0.908 | 0.926 | 0.576 | 0.396 |
| | (8.1%) | | | | | (0.892-0.961) | (0.453-0.696) | |
| White | 7557 | 0.261 | 0.988 | 0.770 | 0.895 | 0.910 | 0.537 | 0.404 |
| | (65.0%) | | | | | (0.898-0.921) | (0.498-0.580) | |
| Black | 1417 | 0.263 | 0.984 | 0.712 | 0.897 | 0.899 | 0.590 | 0.407 |
| | (12.2%) | | | | | (0.872-0.925) | (0.498-0.665) | |
| Other | 1705 | 0.231 | 0.987 | 0.787 | 0.864 | 0.912 | 0.532 | 0.364 |
| | (14.7%) | | | | | (0.887-0.937) | (0.437-0.625) | |
| A-Fib or | 1,518 | 0.274 | 0.974 | 0.572 | 0.912 | 0.858 | 0.540 | 0.421 |
| Flutter | (13.1%) | | | | | (0.831-0.885) | (0.470-0.613) | |
| No A-Fib or | 10,103 | 0.251 | 0.989 | 0.796 | 0.886 | 0.917 | 0.548 | 0.392 |
| Flutter | (86.9%) | | | | | (0.907-0.927) | (0.511-0.586) | |
| Paced ECGs | 551 | 0.360 | 0.983 | 0.302 | 0.987 | 0.821 | 0.626 | 0.528 |
| | (4.7%) | | | | | (0.784-0.858) | (0.549-0.712) | |
| No Paced | 11,070 | 0.241 | 0.988 | 0.786 | 0.873 | 0.908 | 0.527 | 0.378 |
| ECGs | (95.3%) | | | | | (0.898-0.919) | (0.493-0.566) | |
| PR | 1,253 | 0.265 | 0.980 | 0.731 | 0.865 | 0.900 | 0.582 | 0.405 |
| Interval >200 | (10.8%) | | | | | (0.871-0.929) | (0.497-0.671) | |
| PR | 10,368 | 0.255 | 0.988 | 0.773 | 0.896 | 0.911 | 0.540 | 0.398 |
| Interval ≤200 | (89.2%) | | | | | (0.902-0.921) | (0.503-0.579) | |
| LBBB | 399 | 0.328 | 0.953 | 0.277 | 0.963 | 0.804 | 0.602 | 0.489 |
| | (3.4%) | | | | | (0.756-0.852) | (0.504-0.701) | |
| No LBBB | 11,222 | 0.249 | 0.988 | 0.782 | 0.883 | 0.911 | 0.538 | 0.389 |
| | (96.6%) | | | | | (0.901-0.921) | (0.503-0.575) | |
| RBBB | 933 | 0.238 | 0.987 | 0.697 | 0.909 | 0.882 | 0.457 | 0.377 |
| | (8.0%) | | | | | (0.847-0.917) | (0.352-0.547) | |
| No RBBB | 10,688 | 0.259 | 0.988 | 0.775 | 0.890 | 0.912 | 0.554 | 0.401 |
| | (92.0%) | | | | | (0.903-0.922) | (0.519-0.590) | |

* Gender information was not available for 1 patient and age was not available for 3 patient of the total 11,621 patients in the held-out test set
Abbreviations: PPV, positive predictive value; NPV, negative predictive value; AUROC, area under receiver operating characteristic curve; AUPRC, area under precision recall curve; OR, odds ratio; A-Fib, atrial fibrillation; ECG, electrocardiogram; LBBB, left bundle branch block; RBBB, right bundle branch block.

TABLE 4
Model performance in 5-fold cross validation analysis. A 5-fold cross validation
analysis was performed using the original train and validation sets. A patient-
level split stratified by LVEF <40% vs ≥40% was pursued in this analysis
and model performance was assessed on the held-out test set.

| Fold | PPV | NPV | Specificity | Sensitivity | AUROC | AUPRC | F1 Score |
|---|---|---|---|---|---|---|---|
| Fold 1 | 0.242 | 0.988 | 0.748 | 0.898 | 0.905 | 0.520 | 0.381 |
| | | | | | (0.895-0.915) | (0.485-0.556) | |
| Fold 2 | 0.27 | 0.985 | 0.79 | 0.868 | 0.907 | 0.533 | 0.412 |
| | | | | | (0.897-0.916) | (0.499-0.565) | |
| Fold 3 | 0.227 | 0.99 | 0.721 | 0.915 | 0.906 | 0.524 | 0.364 |
| | | | | | (0.897-0.916) | (0.491-0.560) | |
| Fold 4 | 0.269 | 0.987 | 0.786 | 0.882 | 0.910 | 0.543 | 0.413 |
| | | | | | (0.901-0.919) | (0.510-0.576) | |
| Fold 5 | 0.223 | 0.988 | 0.718 | 0.905 | 0.902 | 0.512 | 0.358 |
| | | | | | (0.892-0.912) | (0.479-0.548) | |

Abbreviations: PPV, positive predictive value;
NPV, negative predictive value;
AUROC, area under receiver operating characteristic curve;
AUPRC, area under precision recall curve

### Model Performance Across ECG Formats and Calibrations

The model performance was comparable across the four original layouts of ECG images in the held-out set with AUROC of 0.91 in detecting concurrent LV systolic dysfunction (Table 5). The model had a sensitivity of 0.89 and a positive prediction conferred 26- to 27-fold higher odds of LV systolic dysfunction on the standard and the three variations of the data. In sensitivity analyses, the model demonstrated similar performance in detecting LV systolic dysfunction from novel ECG formats that were not encountered before, with AUROC between 0.88-0.91 (Table 6).

TABLE 5
Performance of model on different image formats created from the held-out test set.

| Format | PPV | NPV | Specificity | Sensitivity | AUROC | AUPRC | F1 Score |
|---|---|---|---|---|---|---|---|
| Standard | 0.257 | 0.988 | 0.769 | 0.892 | 0.910 | 0.545 | 0.399 |
| | | | | | (0.901-0.919) | (0.513-0.581) | |
| Two- | 0.256 | 0.987 | 0.769 | 0.889 | 0.907 | 0.533 | 0.397 |
| Rhythm | | | | | (0.897-0.916) | (0.500-0.566) | |
| Alternate | 0.246 | 0.987 | 0.756 | 0.892 | 0.908 | 0.534 | 0.386 |
| | | | | | (0.899-0.917) | (0.501-0.567) | |
| Shuffled | 0.261 | 0.987 | 0.777 | 0.882 | 0.911 | 0.538 | 0.403 |
| | | | | | (0.902-0.920) | (0.504-0.575) | |

Abbreviations: PPV, positive predictive value; NPV, negative predictive value; AUROC, area under receiver operating characteristic curve; AUPRC, area under precision recall curve.

TABLE 6
Performance of model on novel image formats created from the held-out test
set. Standard format was used both in model training and validation and is
presented for comparison. The three other layouts were only used for validation
to assess model performance on image formats not encountered before.

| Format | PPV | NPV | Specificity | Sensitivity | AUROC | AUPRC | F1 Score |
|---|---|---|---|---|---|---|---|
| Standard | 0.257 | 0.988 | 0.769 | 0.892 | 0.910 | 0.545 | 0.399 |
| | | | | | (0.901-0.919) | (0.513-0.581) | |
| Three-Rhythm | 0.224 | 0.990 | 0.715 | 0.919 | 0.907 | 0.533 | 0.361 |
| | | | | | (0.898-0.917) | (0.503-0.569) | |
| No-Rhythm | 0.204 | 0.988 | 0.684 | 0.907 | 0.887 | 0.465 | 0.333 |
| | | | | | (0.877-0.898) | (0.432-0.500) | |
| Rhythm on Top | 0.220 | 0.987 | 0.715 | 0.897 | 0.901 | 0.504 | 0.353 |
| | | | | | (0.892-0.911) | (0.468-0.540) | |

Abbreviations: PPV, positive predictive value; NPV, negative predictive value; AUROC, area under receiver operating characteristic curve; AUPRC, area under precision recall curve.

The model performance was also consistent across ECG calibrations with an AUROC between 0.88 and 0.91 on ECG calibrations of 5, 10, and 20 mm/mV and AUROC 0.909 (0.900-0.918) and AUPRC of 0.539 (0.504-0.574) with mixed calibrations in the held-out test set. The mixed calibration was generated with a random sample of 5 mm/mV and 20 mm/mV calibrations from the highest and lowest quartiles of voltages, respectively, in lead I (together representing 25% of the sample from the test set), along with 10 mm/mV (remaining 75% of test set) (Table 7). Further sensitivity analyses demonstrated consistent model performance on ECGs (a) without prolonged PR interval (AUROC 0.920 and AUPRC 0.537, Table 8), (b) without paced rhythms (AUROC 0.908, AUPRC 0.519, Table 9), and (c) without atrial fibrillation, atrial flutter, and conduction disorders (AUROC 0.919, AUPRC 0.536, Table 10). Model performance was also consistent across subsets on the held-out test set based on the timing of the ECG relative to the echocardiogram (Table 11).

TABLE 7
Performance of model on standard format held-out test
set images generated from differently calibrated ECGs.

| | | | | | | | F1 |
|---|---|---|---|---|---|---|---|
| Calibration | PPV | NPV | Specificity | Sensitivity | AUROC | AUPRC | Score |
| 5 mm/mV | 0.179 | 0.991 | 0.615 | 0.939 | 0.898 | 0.504 | 0.301 |
| | | | | | (0.888- | (0.470- | |
| | | | | | 0.908) | 0.538) | |
| 10 mm/mV | 0.257 | 0.988 | 0.769 | 0.892 | 0.910 | 0.545 | 0.399 |
| | | | | | (0.901- | (0.509- | |
| | | | | | 0.919) | 0.580) | |
| 20 mm/mV | 0.236 | 0.981 | 0.757 | 0.838 | 0.882 | 0.466 | 0.368 |
| | | | | | (0.871 | (0.432- | |
| | | | | | 0.893) | 0.500) | |
| Mixed* | 0.248 | 0.989 | 0.754 | 0.903 | 0.909 | 0.539 | 0.389 |
| | | | | | (0.900- | (0.504- | |
| | | | | | 0.918) | 0.574) | |

Abbreviations: PPV, positive predictive value; NPV, negative predictive value; AUROC, area under receiver operating characteristic curve; AUPRC, area under precision recall curve.
*The mixed calibration set was generated with a partial sample of 5 mm/mV and 20 mm/mV calibrations from high and low voltages in lead I (together representing 25% of sample from test set), along with 10 mm/mV (remaining 75% of test set)

TABLE 8
Performance of the model in detecting LV systolic
dysfunction from ECGs stratified by PR interval.

| Metric | PR Interval ≤200 | PR Interval >200 |
|---|---|---|
| PPV | 0.247 | 0.265 |
| NPV | 0.990 | 0.981 |
| Specificity | 0.809 | 0.730 |
| Sensitivity | 0.881 | 0.871 |
| AUROC | 0.920 (0.909-0.931) | 0.895 (0.871-0.929) |
| AUPRC | 0.537 (0.502-0.589) | 0.570 (0.495-0.673) |
| F1 Score | 0.390 | 0.405 |

Abbreviations: PPV, positive predictive value; NPV, negative predictive value; AUROC, area under receiver operating characteristic curve; AUPRC, area under precision-recall curve.

TABLE 9
Performance of the model in detecting LV systolic
dysfunction from paced and non-paced ECGs.

| Metric | Non-paced ECGs | Paced ECGs |
|---|---|---|
| PPV | 0.239 | 0.363 |
| NPV | 0.987 | 0.969 |
| Specificity | 0.785 | 0.317 |
| Sensitivity | 0.872 | 0.975 |
| AUROC | 0.908 (0.898-0.919) | 0.817 (0.784-0.858) |
| AUPRC | 0.519 (0.491-0.562) | 0.617 (0.554-0.714) |
| F1 Score | 0.378 | 0.528 |

Abbreviations: PPV, positive predictive value; NPV, negative predictive value; AUROC, area under receiver operating characteristic curve; AUPRC, area under precision recall curve.

TABLE 10
Performance of model on ECGs after exclusion of atrial fibrillation,
atrial flutter, conduction disorders, and paced rhythms.

| Metric | Performance |
|---|---|
| PPV | 0.218 |
| NPV | 0.990 |
| Specificity | 0.840 |
| Sensitivity | 0.840 |
| AUROC | 0.919 (0.905-0.933) |
| AUPRC | 0.536 (0.481-0.585) |
| F1 Score | 0.346 |

Abbreviations: PPV, positive predictive value; NPV, negative predictive value; AUROC, area under receiver operating characteristic curve; AUPRC, area under precision recall curve.

TABLE 11
Performance of the model, stratified by patients with TTE
performed before, after, or on the same day as the ECG.

| | TTE and ECG on | | |
|---|---|---|---|
| | the Same Day | TTE After ECG | TTE Before ECG |
| Performance | N = 2276 | N = 6881 | N = 2464 |
| Metric | (19.6%) | (59.2%) | (21.2%) |
| PPV | 0.277 | 0.232 | 0.291 |
| NPV | 0.983 | 0.990 | 0.985 |
| Specificity | 0.744 | 0.787 | 0.737 |
| Sensitivity | 0.882 | 0.890 | 0.905 |
| AUROC | 0.900 (0.881- | 0.920 (0.908- | 0.894 (0.874- |
| | 0.920) | 0.932) | 0.915) |
| AUPRC | 0.536 (0.468- | 0.534 (0.483- | 0.590 (0.531- |
| | 0.608) | 0.582) | 0.649) |
| F1 Score | 0.422 | 0.368 | 0.441 |

### LV Systolic Dysfunction in Model-Predicted False Positives

Of the 10,666 ECGs in the held-out test set with an associated LVEF≥40% on a proximate echocardiogram, the model classified 2,469 (23.1%) as “false positives”, and 8,197 (76.9%) as true negatives. In further evaluation of false positives, 562 (22.8% of false positives) had evidence of mild LV systolic dysfunction with LVEF between 40-50% on concurrent echocardiography.

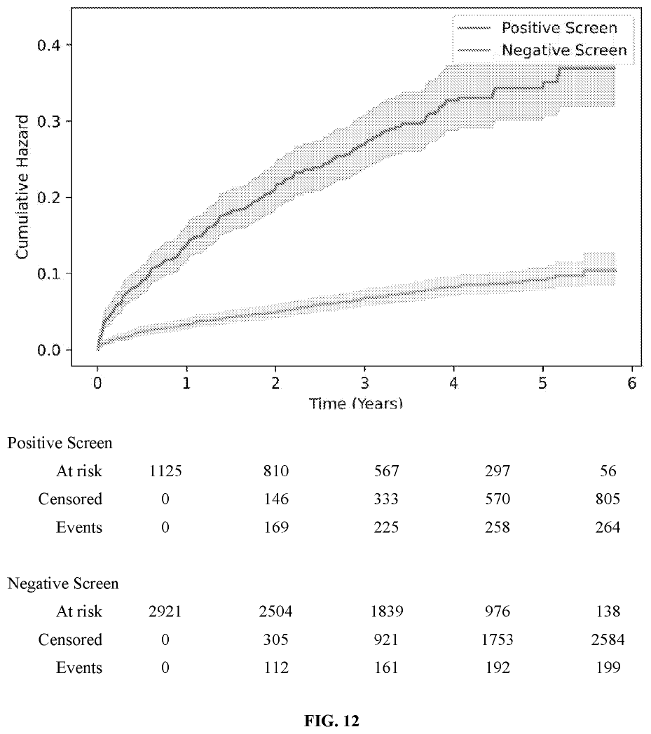

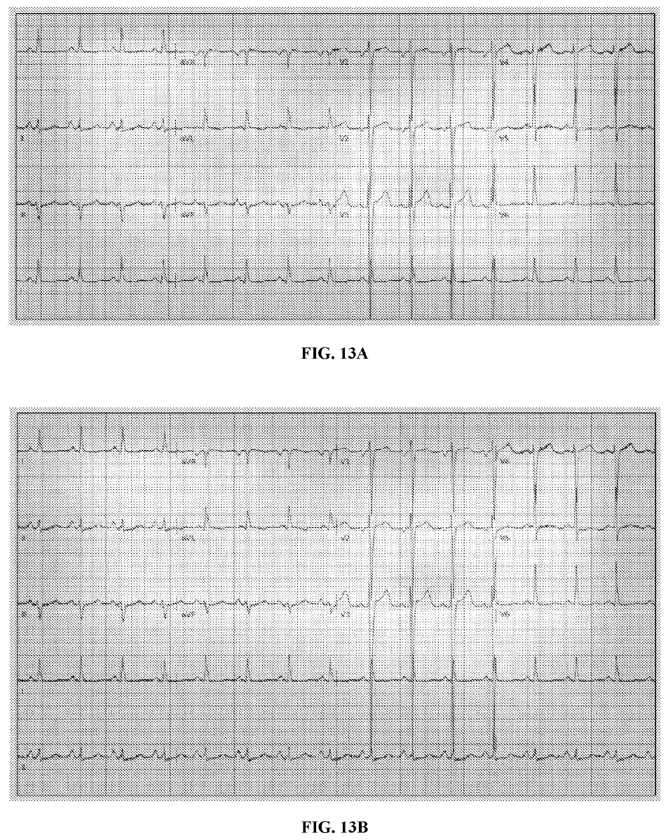

In this group of individuals, 4,046 patients had at least one follow-up TTE, including 1,125 (27.8%) false positives and 2,921 (72.2%) true negatives on the initial index screen. There were 2,665 and 6,083 echocardiograms in the false positive and true negative populations during the follow-up, with the longest follow-up of 6.1 years. Overall, 264 (23.5%) patients with model-predicted positive screen and 199 (6.8%) with negative screen developed new LVEF<40% over the median follow-up of 3.2 years (IQR 1.8-4.4 years, FIG. 12). This represented a 3.9-fold higher risk of incident low LVEF based on having a positive screening result (HR 3.9, 95% CI 3.3-4.7). After adjustment for age, sex, and LVEF at the time of screening, patients with positive screen had a 2.3-fold higher risk of incident low LVEF (Adjusted HR 2.3, 95% CI 1.9-2.8).

### Localization of Predictive Cues for LV Systolic Dysfunction

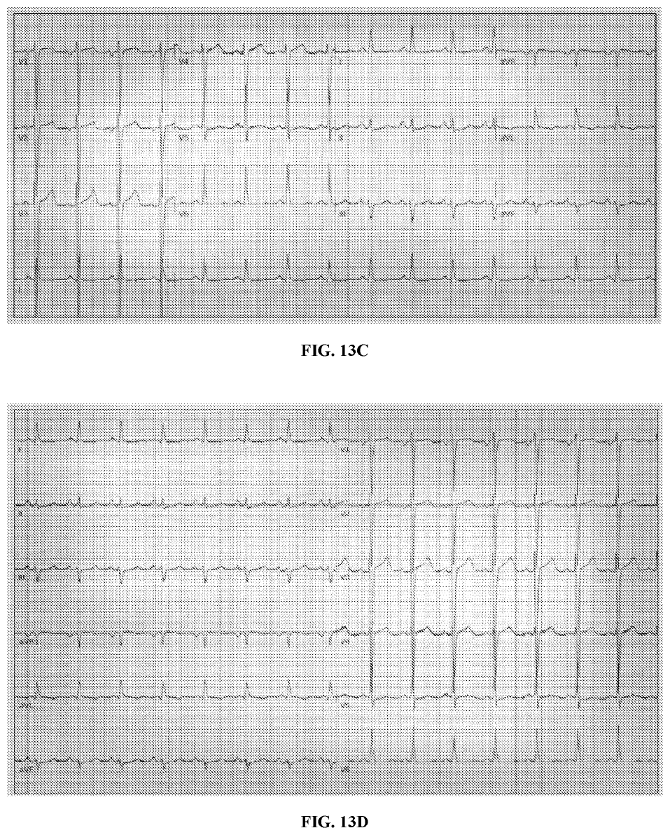

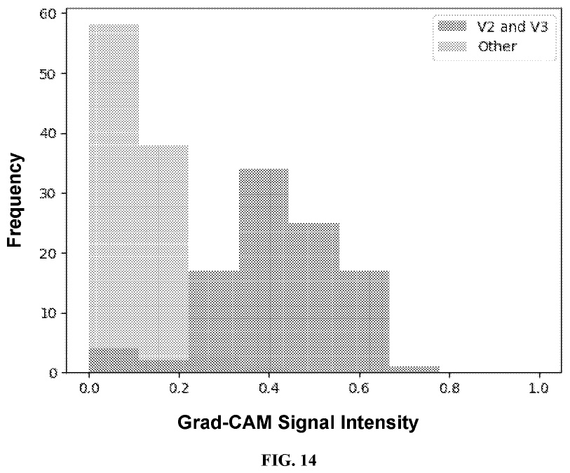

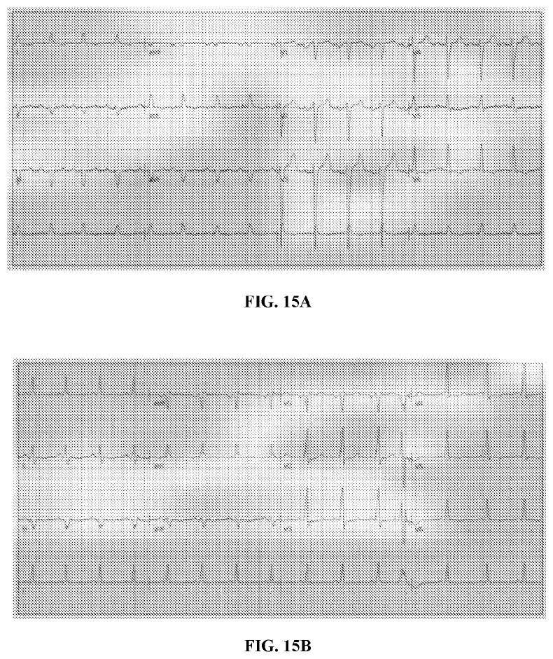

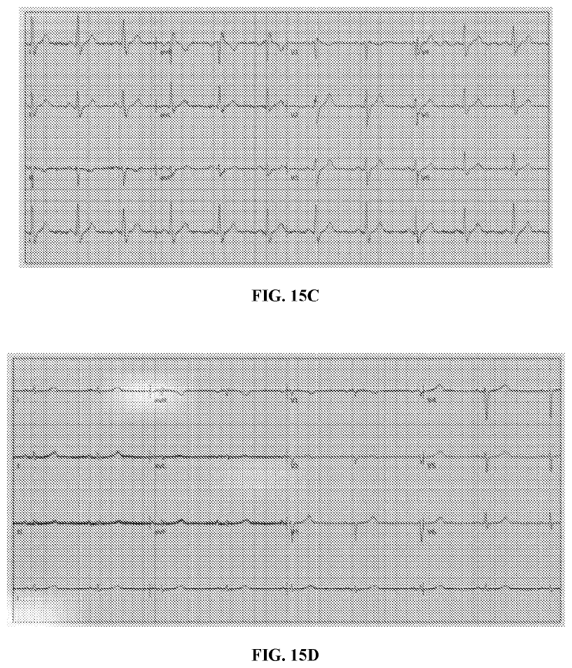

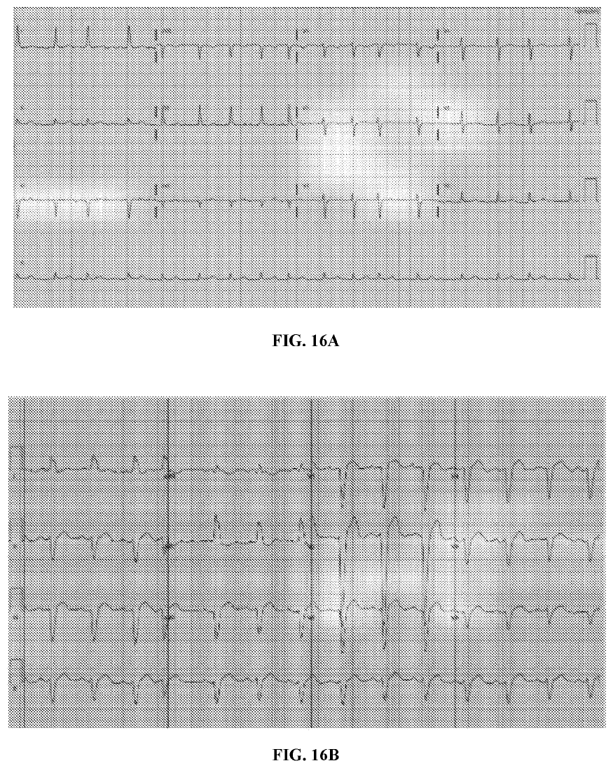

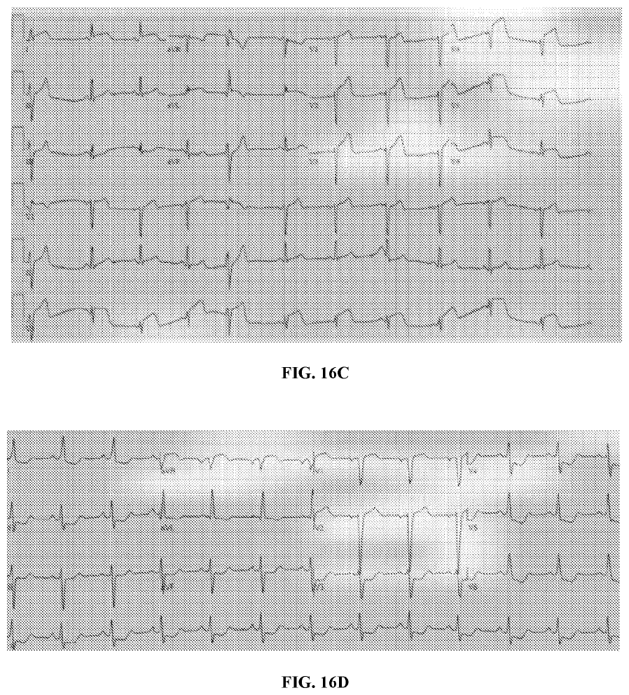

Class activation heatmaps of the 100 positive cases with the most confident model predictions for reduced LVEF prediction across four ECG layouts are presented in FIGS. 13A-D. For all four formats of images, the region corresponding to leads V2 and V3 were the most important areas for prediction of reduced LVEF. FIG. 14 represents the distribution of mean Grad-CAM signal intensities in the regions corresponding to leads V2 and V3 and the other regions of standard format ECGs in this sample. For the majority of cases, the Grad-CAM signal intensities in the V2-V3 area were higher than the other regions of the ECG. Representative images of Grad-CAM analysis in sampled individuals with positive and negative screens in the held-out test set, and non-synthetic ECG images in validation sites are presented in FIGS. 15A-D and 16A-D, respectively.

### External Validation

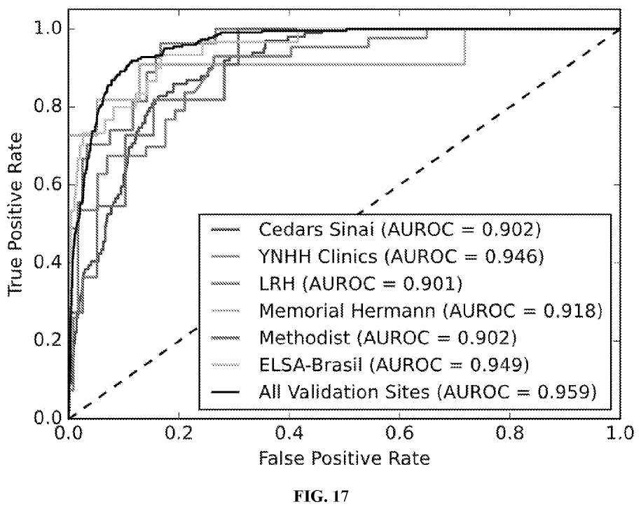

The validation performance of the model was consistent and robust across each of the 6 validation datasets (FIG. 17). The first validation set at Cedars Sinai Medical Center included 879 ECGs from consecutive patients who underwent outpatient echocardiography, including 99 (11%) individuals with LVEF<40%. The model demonstrated an AUROC of 0.90 and an AUPRC of 0.53 in this set. Second, a total of 147 ECG images drawn from YNHH outpatient clinics were used for validation and included 27 images (18%) from patients with LVEF<40%. The model had an AUROC of 0.94 and AUPRC of 0.77 in validation on these images. The third image dataset included ECG images from inpatient visits to the LRH. It included 100 ECG images, with 43 images (43%) from patients with LVEF<40%, with a model AUROC of 0.90 and AUPRC of 0.88. The fourth dataset from Memorial Hermann Southeast Hospital included 50 ECG images, 11 (22%) from patients with LVEF<40%, with a model AUROC and AUPRC of 0.91 and 0.88 on these images, respectively. The fifth validation set contained 50 ECG images from the Methodist Cardiology Clinic, which included 11 (20%) ECGs from patients with LVEF<40%, with model AUROC of 0.90 and AUPRC of 0.74.

The sixth set included 2,577 ECGs from prospectively enrolled individuals in the ELSA-Brasil study, including 30 with LVEF<40%. The model demonstrated an AUROC 0.95 and AUPRC 0.45 on this set. In a mixed sample of ECG-echocardiography data from all external validation sites, the model demonstrated an AUROC and AUPRC of 0.96 (0.950-0.969) and 0.63 (0.563-0.694), respectively, in detecting LV systolic dysfunction, respectively. The model performance on these 6 validation sets is outlined in Tables 12-15.

TABLE 12
Performance of model on external validation datasets.

| Site | PPV | NPV | Specificity | Sensitivity | AUROC | AUPRC | F1 Score |
|---|---|---|---|---|---|---|---|
| Cedars Sinai | 0.326 | 0.979 | 0.772 | 0.869 | 0.902 | 0.533 | 0.474 |
| Medical Center | | | | | (0.877-0.926) | (0.432-0.640) | |
| Outpatient Clinics | 0.338 | 1.000 | 0.558 | 1.000 | 0.946 | 0.775 | 0.505 |
| of YNHH | | | | | (0.910-0.982) | (0.605-0.916) | |
| LRH | 0.538 | 0.955 | 0.368 | 0.977 | 0.901 | 0.889 | 0.694 |
| | | | | | (0.843-0.959) | (0.810-0.946) | |
| Memorial Hermann | 0.385 | 0.958 | 0.590 | 0.909 | 0.918 | 0.888 | 0.541 |
| Southeast Hospital | | | | | (0.790-1.000) | (0.699-1.000) | |
| Methodist | 0.458 | 1.000 | 0.667 | 1.000 | 0.902 | 0.738 | 0.629 |
| Cardiology Clinic | | | | | (0.816-0.989) | (0.470-0.928) | |
| ELSA-Brasil | 0.256 | 0.996 | 0.976 | 0.700 | 0.949 | 0.449 | 0.375 |
| | | | | | (0.915-0.983) | (0.290-0.651) | |
| All Validation | 0.356 | 0.993 | 0.900 | 0.891 | 0.959 | 0.631 | 0.508 |
| Sites | | | | | (0.950-0.969) | (0.563-0.694) | |

Abbreviations: PPV, positive predictive value;
NPV, negative predictive value;
AUROC, area under receiver operating characteristic curve;
AUPRC, area under precision recall curve;
ELSA-Brasil, Estudo Longitudinal de Saúde do Adulto (The Brazilian Longitudinal Study of Adult Health)

TABLE 13
Model prediction scores for individuals in external
validation sets across the LV ejection fraction.

| | Ejection | Prediction Score | | | |
|---|---|---|---|---|---|
| Validation Site | Fraction | 0-0.1 | 0.1-0.2 | 0.2-0.5 | 0.5-1 |
| Cedars Sinai | <40% | 13 | 11 | 35 | 40 |
| Medical Center | | (13.1%) | (11.1%) | (35.4%) | (40.4%) |
| | 40-50% | 36 | 16 | 29 | 16 |
| | | (37.1%) | (16.5%) | (29.9%) | (16.5%) |
| | >50% | 566 | 54 | 44 | 19 |
| | | (82.9%) | (7.9%) | (6.4%) | (2.8%) |
| Outpatient | <40% | 0 | 0 | 7 | 20 |
| YNHH Clinics | | (0%) | (0%) | (25.9%) | (74.1%) |
| | 40-50% | 2 | 2 | 6 | 4 |
| | | (14.3%) | (14.3%) | (42.9%) | (28.6%) |
| | >50% | 65 | 13 | 18 | 10 |
| | | (61.3%) | (12.3%) | (17.0%) | (9.4%) |
| LRH | <40% | 1 | 2 | 15 | 25 |
| | | (2.3%) | (4.7%) | (34.9%) | (58.1%) |
| | 40-50% | 4 | 6 | 4 | 2 |
| | | (25.0%) | (37.5%) | (25.0%) | (12.5%) |
| | >50% | 17 | 11 | 12 | 1 |
| | | (41.4%) | (26.8%) | (29.3%) | (2.4%) |
| Memorial | <40% | 1 | 0 | 1 | 9 |
| Hermann | | (9.1%) | (0%) | (9.1%) | (81.8%) |
| Southeast | 40-50% | 5 | 2 | 1 | 1 |
| Hospital | | (55.6%) | (22.2%) | (11.1%) | (11.1%) |
| | >50% | 18 | 4 | 7 | 1 |
| | | (60.0%) | (13.3%) | (23.3%) | (3.3%) |
| Methodist | <40% | 0 | 2 | 3 | 6 |
| Cardiologist | | (0%) | (18.2%) | (27.3%) | (54.5%) |
| Clinic | 40-50% | 5 | 0 | 0 | 1 |
| | | (83.3%) | (0%) | (0%) | (16.7%) |
| | >50% | 21 | 2 | 8 | 2 |
| | | (63.6%) | (6.1%) | (24.2%) | (6.1%) |
| ELSA-Brasil | <40% | 9 | 4 | 5 | 12 |
| | | (30.0%) | (13.3%) | (16.7%) | (40.0%) |
| | 40-50% | 32 | 2 | 5 | 3 |
| | | (76.2%) | (4.8%) | (11.9%) | (7.1%) |
| | >50% | 2454 | 29 | 19 | 3 |
| | | (98.0%) | (1.2%) | (0.7%) | (0.1%) |

Abbreviations:
EF, Ejection fraction;
ELSA-Brasil, Estudo Longitudinal de Saúde do Adulto (The Brazilian Longitudinal Study of Adult Health);
LRH, Lake Regional Hospital;
YNHH, Yale New Haven Hospital

TABLE 14
Confusion matrices for model performance on
real-world external validation datasets.

| | True | False | True | False |
|---|---|---|---|---|
| Validation Site | Positive | Positive | Negative | Negative |
| Cedars Sinai Medical Center | 86 | 178 | 602 | 13 |
| Outpatient YNHH Clinics | 27 | 53 | 67 | 0 |
| LRH | 42 | 36 | 21 | 1 |
| Memorial Hermann Southeast | 10 | 16 | 23 | 1 |
| Hospital | | | | |
| Methodist Cardiologist Clinic | 11 | 13 | 26 | 0 |
| ELSA-Brasil | 21 | 61 | 2486 | 9 |

Abbreviations:
ELSA-Brasil, Estudo Longitudinal de Saúde do Adulto (The Brazilian Longitudinal Study of Adult Health);
LRH, Lake Regional Hospital;
YNHH, Yale New Haven Hospital

TABLE 15
Assessment of Model Performance Using Different Cut-off Values
in the Held-out Test Set and External Validation Sites.

| Site | Cut-off | PPV | NPV | Specificity | Sensitivity |
|---|---|---|---|---|---|
| Held-out | 0.05 | 0.196 | 0.991 | 0.658 | 0.934 |
| Test Set | 0.1 | 0.256 | 0.988 | 0.768 | 0.892 |
| | 0.2 | 0.359 | 0.98 | 0.871 | 0.806 |
| | 0.3 | 0.426 | 0.972 | 0.915 | 0.705 |
| | 0.5 | 0.535 | 0.958 | 0.959 | 0.532 |
| Cedars Sinai | 0.05 | 0.254 | 0.988 | 0.65 | 0.939 |
| Medical | 0.1 | 0.326 | 0.979 | 0.772 | 0.869 |
| Center | 0.2 | 0.41 | 0.966 | 0.862 | 0.758 |
| | 0.3 | 0.437 | 0.946 | 0.903 | 0.596 |
| | 0.5 | 0.533 | 0.927 | 0.955 | 0.404 |
| Outpatient | 0.05 | 0.255 | 1 | 0.342 | 1 |
| YNHH | 0.1 | 0.338 | 1 | 0.558 | 1 |
| Clinics | 0.2 | 0.415 | 1 | 0.683 | 1 |
| | 0.3 | 0.553 | 0.99 | 0.825 | 0.963 |
| | 0.5 | 0.588 | 0.938 | 0.883 | 0.741 |
| LRH | 0.05 | 0.506 | 1 | 0.263 | 1 |
| | 0.1 | 0.538 | 0.955 | 0.368 | 0.977 |
| | 0.2 | 0.678 | 0.927 | 0.667 | 0.93 |
| | 0.3 | 0.767 | 0.825 | 0.825 | 0.767 |
| | 0.5 | 0.893 | 0.75 | 0.947 | 0.581 |
| Memorial | 0.05 | 0.278 | 0.929 | 0.333 | 0.909 |
| Hermann | 0.1 | 0.385 | 0.958 | 0.59 | 0.909 |
| Southeast | 0.2 | 0.5 | 0.967 | 0.744 | 0.909 |
| Hospital | 0.3 | 0.643 | 0.944 | 0.872 | 0.818 |
| | 0.5 | 0.818 | 0.949 | 0.949 | 0.818 |
| Methodist | 0.05 | 0.333 | 1 | 0.436 | 1 |
| Cardiologist | 0.1 | 0.458 | 1 | 0.667 | 1 |
| Clinic | 0.2 | 0.45 | 0.933 | 0.718 | 0.818 |
| | 0.3 | 0.571 | 0.917 | 0.846 | 0.727 |
| | 0.5 | 0.667 | 0.878 | 0.923 | 0.545 |
| ELSA- | 0.05 | 0.123 | 0.997 | 0.938 | 0.733 |
| Brasil | 0.1 | 0.256 | 0.996 | 0.976 | 0.7 |
| | 0.2 | 0.362 | 0.995 | 0.988 | 0.567 |
| | 0.3 | 0.485 | 0.994 | 0.993 | 0.533 |
| | 0.5 | 0.667 | 0.993 | 0.998 | 0.4 |
| All | 0.05 | 0.256 | 0.995 | 0.833 | 0.932 |
| Validation | 0.1 | 0.356 | 0.993 | 0.9 | 0.891 |
| Sites | 0.2 | 0.452 | 0.987 | 0.94 | 0.805 |
| | 0.3 | 0.528 | 0.98 | 0.962 | 0.683 |
| | 0.5 | 0.64 | 0.97 | 0.982 | 0.507 |

Abbreviations:
ELSA-Brasil, Estudo Longitudinal de Saúde do Adulto (The Brazilian Longitudinal Study of Adult Health);
LRH, Lake Regional Hospital;
NPV, Negative Predictive Value;
PPV, Positive Predictive Value;
YNHH, Yale New Haven Hospital

### Quality Assurance in Real World Applications

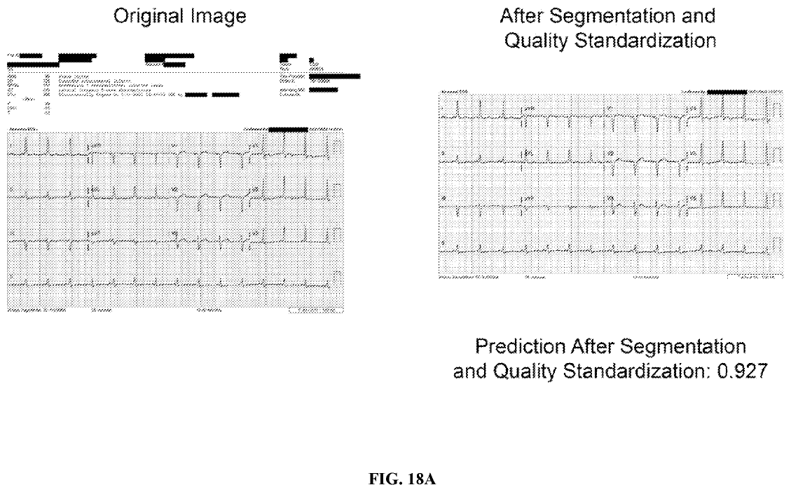

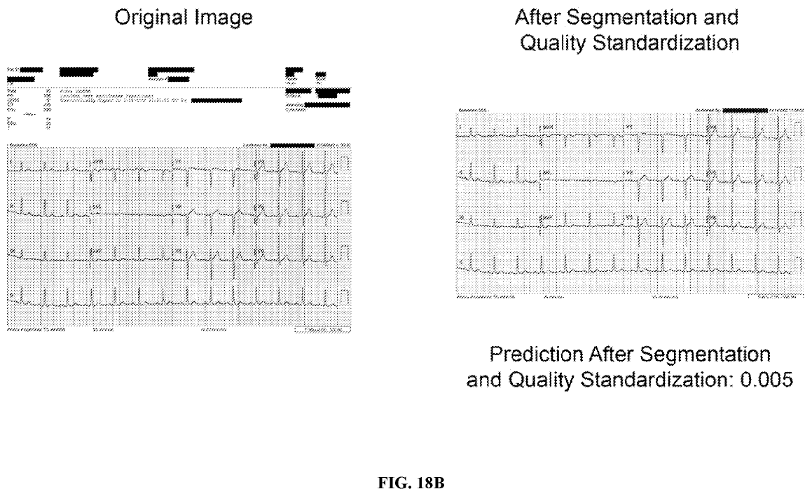

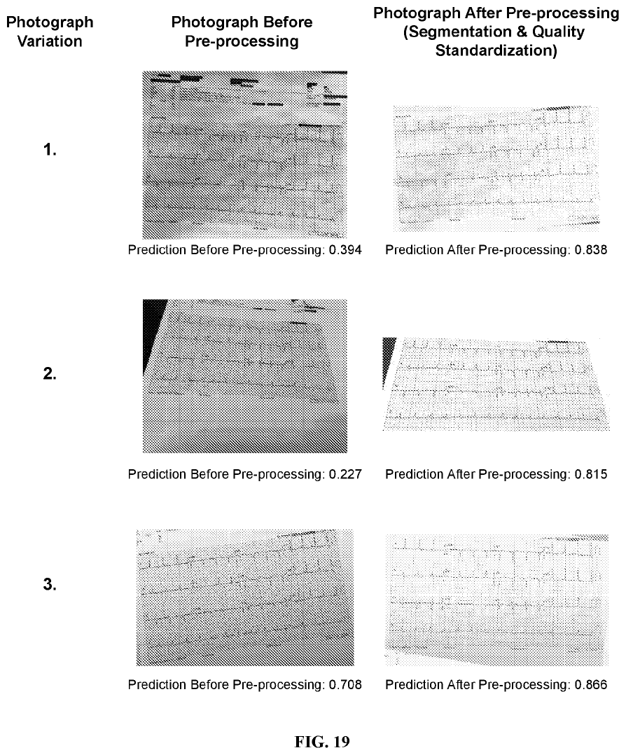

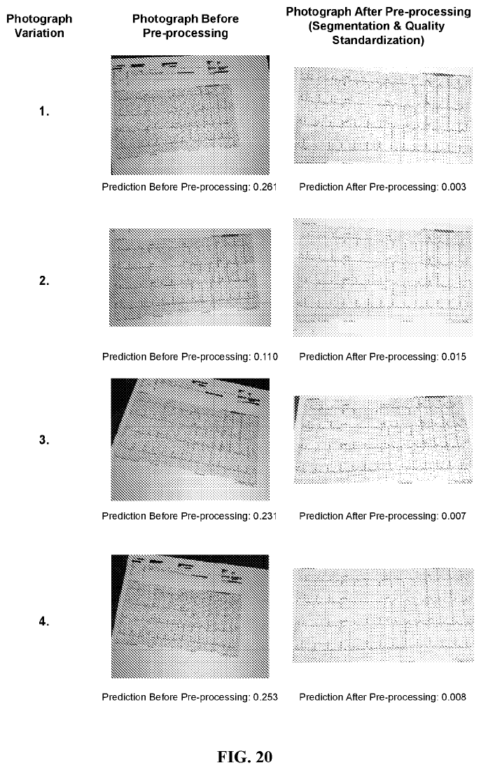

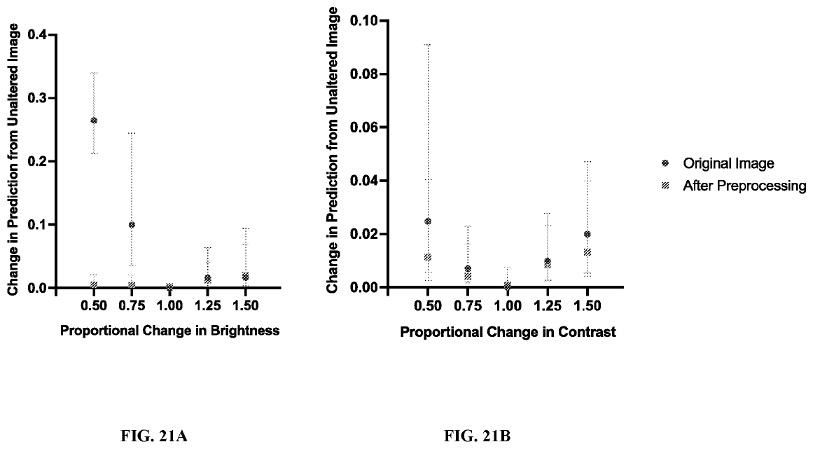

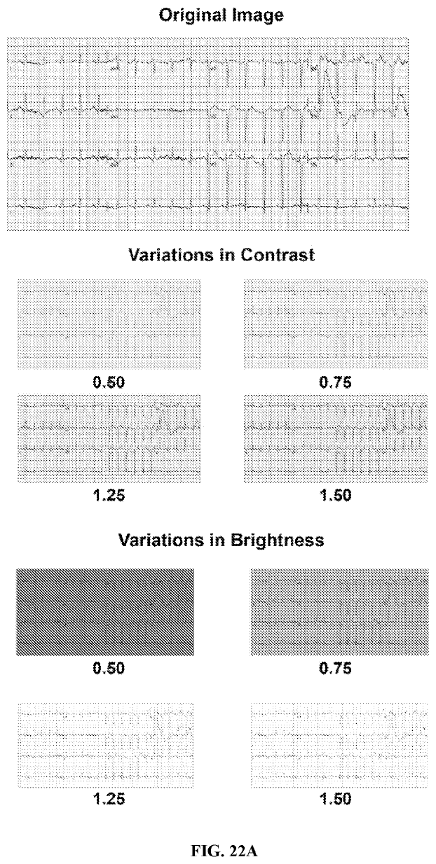

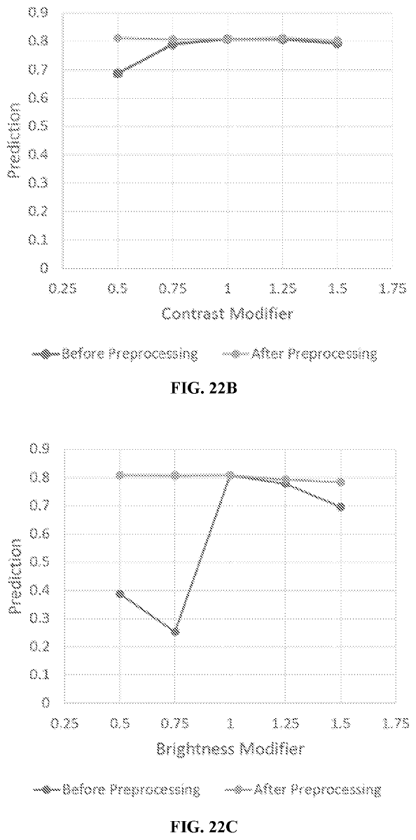

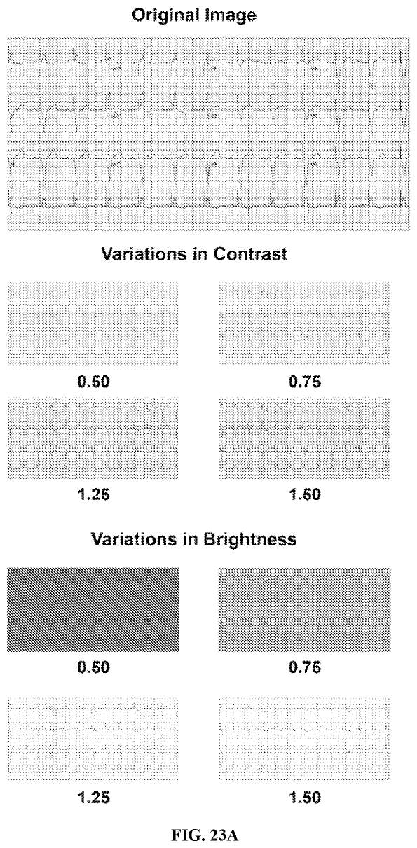

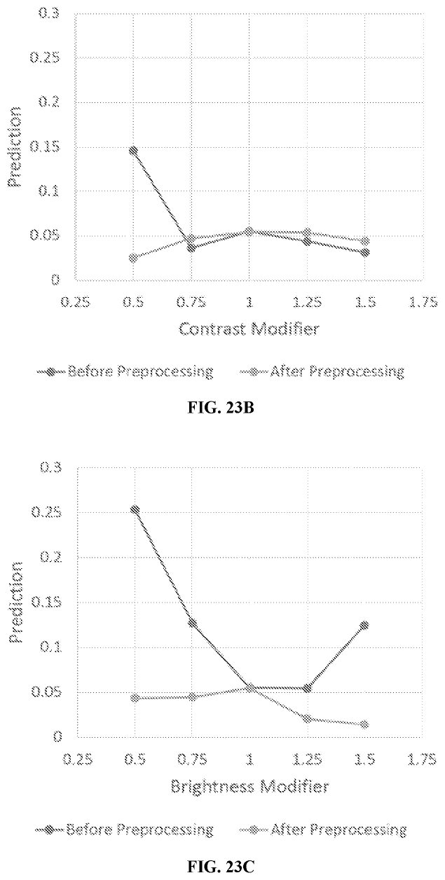

We assessed our preprocessing pipeline in segmentation and quality standardization of real-world ECG images. FIGS. 18A-B represents examples of ECGs in electronic PDF format before and after preprocessing and demonstrates the automated removal of ECG annotations and patient identifiers from the image. FIGS. 19 and 20 demonstrate quality standardization of photographs of ECGs obtained by a smartphone with extreme variations of photo brightness, shadows, skew angles, and noise artifacts. Furthermore, we systematically evaluated our model calibration across the variations of photo brightness and contrast in a sample of 200 ECGs randomly selected from the held-out test set in a 1:4 ratio for LVEF<4000 and >40%, respectively. We observed minimal changes in model-predicted probabilities despite 50% alterations in image brightness and contrast on preprocessed images (FIG. 21A-B). This effect remained consistent across ECGs from individuals with low (FIG. 22A-B) and normal (FIG. 23A-B) LVEF. Table 16 presents the confusion matrices for model predictions at varying levels of input image brightness and contrast, with or without preprocessing.

TABLE 16
Confusion Matrices for Model Predictions of LV systolic dysfunction at Varying
Levels of Brightness and Contrast, with or without preprocessing

| | | Without Preprocessing | | | | With Preprocessing | | | |
|---|---|---|---|---|---|---|---|---|---|
| | | True | False | True | False | True | False | True | False |
| Characteristic | | Positive | Positive | Negative | Negative | Positive | Positive | Negative | Negative |
| Brightness | 0.50 | 40 | 160 | 0 | 0 | 39 | 31 | 129 | 1 |
| | 0.75 | 39 | 74 | 86 | 1 | 39 | 31 | 129 | 1 |
| | 1.00 | 39 | 29 | 131 | 1 | 39 | 29 | 131 | 1 |
| | 1.25 | 38 | 23 | 137 | 2 | 38 | 31 | 129 | 2 |
| | 1.50 | 38 | 23 | 137 | 2 | 38 | 35 | 125 | 2 |
| Contrast | 0.50 | 39 | 40 | 120 | 1 | 39 | 33 | 127 | 1 |
| | 0.75 | 39 | 25 | 135 | 1 | 39 | 29 | 131 | 1 |
| | 1.00 | 39 | 29 | 131 | 1 | 39 | 29 | 131 | 1 |
| | 1.25 | 39 | 38 | 122 | 1 | 39 | 37 | 123 | 1 |
| | 1.50 | 39 | 40 | 120 | 1 | 40 | 41 | 119 | 0 |

### DISCUSSION

We developed and externally validated an automated deep learning algorithm that accurately identifies LV systolic dysfunction solely from ECG images. The algorithm has high discrimination and sensitivity, representing characteristics ideal for a screening strategy. It is robust to variations in the layouts of ECG waveforms and detects the location of ECG leads across multiple formats with consistent accuracy, making it suitable for implementation in a variety of settings. Moreover, the algorithm was developed and tested in a diverse population with high performance in subgroups of age, sex, and race, and across geographically dispersed academic and community health systems. It performed well in 6 external validation sites, spanning both clinical settings as well as a prospective cohort study where protocolized echocardiograms were performed concurrently with ECGs. An evaluation of the class-discriminating signals localized it to the anteroseptal and anterior leads regardless of the ECG layout, topologically corresponding to the left ventricle. Finally, among individuals who did not have a concurrently recorded low LVEF, a positive ECG screen was associated with a 3.9-fold increased risk of developing LV systolic dysfunction in the future compared with those with negative screen, which was significant after adjustment for age, sex, and baseline LVEF. Therefore, an ECG image-based approach can represent a screening as well as predictive strategy for LV systolic dysfunction, particularly in low-resource settings.

Deep learning-based analysis of ECG images to screen for heart failure represents a novel application of AI to improve clinical care. Convolutional neural networks have previously been designed to detect low LVEF from ECG signals. Although reliance of signal-based models on voltage data is not computationally limited, their use in both retrospective and prospective settings requires access to a signal repository where the ECG data architecture varies by ECG device vendors. Moreover, data are often not stored beyond generating printed ECG images, particularly in remote settings. Furthermore, widespread adoption of signal-based models is limited by the implementation barriers requiring health system-wide investments to incorporate them into clinical workflow, something that may not be available or cost-effective in low-resource settings and, to date, is not widely available in higher resource setting such as the US. The algorithm reported in this study overcomes these limitations by making detection of LV systolic dysfunction from ECGs interoperable across acquisition formats and directly available to clinicians who only have access to ECG images. Since scanned ECG images are the most common format of storage and use of electrocardiograms, untrained operators can implement large scale screening through chart review or automated applications to image repositories—a lower resource task than optimizing tools for different machines.

The use of ECG images in our model overcomes the implementation challenges arising from black box algorithms. The origin of risk-discriminative signals in precordial leads of ECG images suggests a left ventricular origin of the predictive signals. Moreover, the consistent observation of these predictive signals in the anteroseptal and anterior leads, regardless of the lead location on printed images, also serves as a control for the model predictions. Despite localizing the class-discriminative signals in the image to the left ventricle, heatmap analysis may not necessarily capture all the model predictive features, such as the duration of ECG segments, intervals, or ECG waveform morphologies that might have been used in model predictions. However, visual representations consistent with clinical knowledge could explain parts of the model prediction process and address the hesitancy in the uptake of these tools in clinical practice.

An important finding was the significantly increased risk of incident LV systolic dysfunction among patients with model-predicted positive screen but LVEF≥40% on concurrent echocardiography. These findings demonstrate an electrocardiographic signature that may precede the development of echocardiographic evidence of LV systolic dysfunction. This was previously reported in signal-based models, further suggesting that the detection of LV systolic dysfunction on ECG images represents a similar underlying pathophysiological process. Moreover, we observed a linear relationship between the severity of LV systolic dysfunction and the model-predicted probabilities of low LVEF, supporting the biological plausibility of model predictions from paired ECG and echocardiography data. These observations suggest a role for AI-based ECG models in risk stratification for cardiovascular disease.

Our model's ability to consistently distinguish LV systolic dysfunction across demographic subgroups and validation populations suggests robustness and generalizability of the effects though prospective assessments in the intended screening setting are warranted. Notably, the model demonstrated a higher specificity and lower sensitivity on the ELSA-Brasil cohort composed of younger and generally healthier individuals with a lower prevalence of LV systolic dysfunction compared to the other validation sets. Depending on the intended result of the screening approach and resource constraints with downstream testing, prediction thresholds for LV systolic dysfunction may need to be recalibrated when deployed in such settings. While the model development pursues preprocessing the ECG signal for plotting images, when preprocessing is performed before ECG images are generated and/or printed by ECG machines further processing of images is not required for real-world application, as demonstrated in the application of the model to the external validation sets.

### CONCLUSIONS

We developed an automated algorithm to detect LV systolic dysfunction from ECG images, demonstrating a robust performance across subgroups of patient demographics, ECG formats and calibrations, and clinical practice settings. Given the ubiquitous availability of ECG images, this approach represents a strategy for automated screening of LV systolic dysfunction, especially in resource-limited settings.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method of detecting cardiovascular disease in a subject, the method comprising:
receiving an electrocardiogram (ECG) image for the subject;
applying a machine-learning based algorithm to the ECG image for the subject, the algorithm being trained to distinguish a printed ECG reading of a heart with cardiovascular disease from a printed ECG reading of a healthy heart;
comparing outputs of the algorithm to patterns of algorithm outputs for ECG images from healthy subjects and subjects with one or more cardiovascular diseases; and
determining if the subject has cardiovascular disease based upon the outputs of the algorithm.

2. The method of claim 1, wherein the machine-learning based algorithm is a deep neural network, the deep neural network comprising a plurality of nodes trained to distinguish a printed ECG reading of a heart with cardiovascular disease from a printed ECG reading of a healthy heart.

3. The method of claim 1, wherein the machine-learning based algorithm is a statistical algorithm.

4. The method of claim 1, wherein the ECG image comprises a printed ECG image of an ECG dataset formed by conversion of ECG waveform data.

5. The method of claim 1, wherein the method is generalizable to multiple ECG image formats.

6. The method of claim 1, wherein the algorithm trained on ECG images having incorrectly placed leads.

7. The method of claim 1, wherein the algorithm is trained on images of ECGs with different signal, background, and noise characteristics.

8. The method of claim 1, further comprising identifying hidden clinical labels.

9. The method of claim 1, further comprising identifying characteristics of the ECG image that the determination is based on.

10. The method of claim 1, wherein the method is automated.

11. The method of claim 1, wherein the cardiovascular disease comprises a disorder selected from the group consisting of structural disorders of the heart, functional disorders of the heart, structural disorders of the structures supporting the heart, functional disorders of the structures supporting the heart, and combinations thereof.

12. The method of claim 1, wherein the disorder comprises abnormalities of the muscle, valves, blood vessels, or lining of the heart.

13. The method of claim 1, wherein the disorder is a genetic disorder.

14. The method of claim 1, wherein the disorder is an acquired disorder.

15. The method of claim 1, wherein the cardiovascular disease comprises a disease that is not normally discernable by physicians from ECG data.

16. The method of claim 1, wherein, prior to the step of applying the algorithm to the ECG image for the subject, the method further comprises training the algorithm, the training of the algorithm comprising:
creating an image-based dataset including a normal subset and a cardiovascular disease subset;
optionally pre-training the algorithm on an unrelated clinical or hidden label; and
training the algorithm on the image-based dataset.

17. The method of claim 16, wherein the cardiovascular disease subset includes a low ejection fraction (EF) subset.

18. The method of claim 17, wherein the low EF subset includes ECG images for individuals with EF of less than 40%.

19. The method of claim 16, wherein the clinical label includes six physician-defined labels and the hidden label includes gender.

20. The method of claim 16, wherein the normal subset includes ECG images for individuals having hypertrophic cardiomyopathy (HCM).

21. The method of claim 20, wherein the cardiovascular disease subset includes ECG images for individuals having HCM and left ventricular (LV) systolic dysfunction.

22. The method of claim 16, wherein the image-based dataset includes at least two different plotting schemes for each ECG waveform.

23. The method of claim 16, wherein the image-based dataset includes at least two different ECG image formats.
